(19) United States
(12) Patent Application Publication
(10) Pub. No.: US 2026/0301866 A1
(43) Pub. Date: Oct. 1, 2026

(54) Computational Method to Identify Mutational Epistasis in Proteins

(71) Applicants: Paul Campitelli, Tempe, AZ (US); Nicholas Ose, Ramsey, MN (US); Tushar Modi, Woburn, MA (US); Ismail Can Kazan, Tempe, AZ (US); Sefika Banu Ozkan, Phoenix, AZ (US)

(72) Inventors: Paul Campitelli, Tempe, AZ (US); Nicholas Ose, Ramsey, MN (US); Tushar Modi, Woburn, MA (US); Ismail Can Kazan, Tempe, AZ (US); Sefika Banu Ozkan, Phoenix, AZ (US)

(73) Assignee: Arizona Board of Regents on Behalf of Arizona State University, Scottsdale, AZ (US)

(21) Appl. No.: 19/632,102

(22) Filed: Mar. 27, 2026

Related U.S. Application Data
(60) Provisional application No. 63/779,751, filed on Mar. 28, 2025.

(51) Int. Cl. G16B 20/20; G16B 5/00; G16B 15/30

(52) U.S. Cl. CPC G16B 20/20; G16B 5/00; G16B 15/30

(57) ABSTRACT

Systems and methods for characterizing a protein are disclosed herein. The method may include: accessing simulated protein structure data with a computer system, in which the simulated protein structure data indicate a structure of the protein; selecting, using the computer system, a plurality of residues in the protein and quantifying a plurality of dynamics metrics corresponding to each residue in the plurality of residues, in which the dynamics metrics comprise a metric of epistasis, in which the metric of epistasis for a given residue in the plurality of residues is indicative of coupling additivity and non-additivity between the given residue and a pair of residues that does not comprise the given residue; identifying, using the computer system, at least one residue of interest based on the plurality of dynamics metrics; and characterizing, using the computer system, the protein and the at least one residue of interest based on the plurality of dynamics metrics.

### CROSS-REFERENCE TO RELATED APPLICATIONS

This application claims the benefit of and priority to U.S. Application No. 63/779,751, filed Mar. 28, 2025, the entirety of which is incorporated herein by reference.

### STATEMENT REGARDING FEDERALLY SPONSORED RESEARCH OR DEVELOPMENT

This invention was made with government support under 1715591 and 1901709 awarded by the National Science Foundation and GM147635 awarded by the National Institutes of Health. The government has certain rights in the invention.

### BACKGROUND

The SARS-CoV-2 virus has rapidly evolved since its emergence, leading to the emergence of numerous variants of concern (VOCs). Understanding the molecular mechanisms driving these adaptations is crucial for developing effective antiviral strategies. The spike protein, responsible for viral entry into host cells, has been a primary target for research. Previous studies have focused on identifying adaptive mutations and their impact on protein structure and function. However, the role of protein dynamics in viral evolution remains largely unexplored. Thus, there exists a need for a computational tool that quantifies the epistatic interactions between pairs of mutations in the spike protein through a dynamic coupling index (DCI) approach.

### SUMMARY

Disclosed herein are methods and systems for characterizing protein variants. In some embodiments, the systems and methods including: accessing simulated protein structure data with a computer system, in which the simulated protein structure data indicate a structure of the protein; using the computer system to: select a plurality of residues in the protein and to quantify a plurality of dynamics metrics corresponding to each residue in the plurality of residues, in which the dynamics metrics comprise at least a metric of epistasis, in which the metric of epistasis for a given residue in the plurality of residues is indicative of coupling additivity or non-additivity between the given residue and a pair of residues that does not comprise the given residue; identify at least one residue of interest based on the plurality of dynamics metrics; and characterize the protein and the at least one residue of interest based on the plurality of dynamics metrics.

In some embodiments, accessing the simulated protein structure data includes simulating the structure of the protein with the computer system using molecular dynamics, or accessing the structure data that was previously simulated. In some embodiments, the plurality of dynamics metrics further includes at least one of a dynamic flexibility index, a dynamic coupling index, or an asymmetric dynamic coupling index.

In some embodiments, quantifying the metric of epistasis for a given residue further includes: perturbing each residue in the pair of residues individually and quantifying a response in the given residue; perturbing each residue in the pair of residues simultaneously and quantifying the response in the given residue; and calculating the metric of epistasis based on the responses to the individual and the simultaneous perturbations. A “given residue” refers to a specific residue in a protein or peptide.

In some embodiments, the method further includes using the computer system to access evolutionary information of the protein and to calculate an evolutionary probability score for each residue in the plurality of residues. In some embodiments, the method further includes identifying at least one candidate adaptive polymorphism in the plurality of residues based on the evolutionary probability scores. In some embodiments, the method further includes comparing the residues of interest and candidate adaptive polymorphism to determine mechanistic or epistatic relationships between the at least one candidate adaptive polymorphism and the at least one residue of interest.

In some embodiments, the protein includes a protein from a virus. In some embodiments, the method further includes comparing the characterization of the protein and the at least one residue of interest for the protein and the protein from the virus variant.

In some embodiments, the method further includes predicting a functional behavior of the virus based on the plurality of dynamics metrics of each residue in the plurality of residues, and based on the at least one residue of interest.

In some embodiments, the method further includes generating a report characterizing: the protein, the plurality of residues and the corresponding dynamics metrics, and the at least one residue of interest and the corresponding dynamics metrics.

In some embodiments, the method may be executed by a computer system or a non-transitory computer-readable medium.

### BRIEF DESCRIPTION OF THE DRAWINGS

FIGS. 1A-1C show evolutionary probability (EP) in SARS-CoV-2. (FIG. 1A) The EPs of each amino acid in the S protein sequence are calculated by taking the multiple sequence alignment of the S proteins through their evolutionary tree and using Bayesian inferences to determine the likelihood of finding a particular residue at a particular location within a given sequence. Simply, if the residue is found at a location ‘x’ in closely related sequences, it will have a higher EP at location ‘x’ in the target sequence. Residues with an EP<0.05 in the target sequence are candidate adaptive polymorphisms (CAPs) (red). CAPs are found at sites 32, 50, 218, 346, 372, 478, 486, 498, 519, 604, 681, 682, 683, 684, and 1125. (FIG. 1B) The distribution of EP scores of the wild-type residues in the S protein. Here, lower EP scores are shown in red, and higher EP scores in blue. While the vast majority of the wild-type (reference) protein consists of high EP residues, a few residues have low EP. (FIG. 1C) The CAPs are also highlighted as red spheres in the open configuration of the S protein, with the open chain in a darker shade. We observe that a majority of the CAP positions reside at the receptor-binding domain (RBD) and the Furin cleavage site (676-689; Wrobel et al., 2020) shown as transparent light gray spheres.

FIGS. 2A-2B show candidate adaptive polymorphisms (CAPs) control binding sites across chains. (FIG. 2A) Schematic representation of DCI_{asym}. (FIG. 2B) DCI_{asym} of CAP residue positions with the binding interface of receptor-binding domain (RBD) in the open chain. Residues in the closed chains with a low evolutionary probability (EP) amino acid in the reference sequence dominate the binding site interface of RBD in the open chain. There is a significant difference between the asymmetry profiles of the closed (M=−0.06, SD=0.33) and open (M=−1.68, SD=0.89) conformations (p<0.001).

FIG. 3 shows the average number of variants observed among residues of different flexibility. Residues were sorted into one of five bins based on flexibility. Then, the average number of variants for residues within that bin was calculated. Here, the number of variants is defined as the number of different amino acid varieties found at that site. Mutational data was calculated across approximately 24,000 SARS-CoV-2 S protein sequences from the NCBI Datasets Project (Brister et al., 2015). Residue flexibility, as reported here via % DFI, was computed using PDB id 6vsb from the Protein DataBank (Berman et al., 2000). More rigid residues tend to have fewer variants (r=0.94).

FIGS. 4A-4B show mutations in variants of concern (VOCs) present different asymmetry profiles. (FIG. 4A) DCI_{asym} with low evolutionary probability (EP) characteristic mutation sites of Delta or Omicron strains in the closed chains and the binding interface of receptor-binding domain (RBD) in the open chain. Delta displays a second peak closer to zero, suggesting that Delta mutation sites (M=−0.98, SD=0.80) have less allosteric control over the hACE2 binding sites than Omicron mutation sites (M=−1.74, SD=1.00) (p<0.001). However, both sets of sites have far more control over hACE2 binding sites than expected, based on a random control group (M=0.03, SD=0.85) (p<0.001). (FIG. 4B) S protein structure showing binding interface sites (transparent gray), Delta mutation sites (magenta), Omicron mutation sites (cyan), and sites mutated in both Omicron and Delta (blue).

FIGS. 5A-5B show EpiScore provides a measurement of epistasis. (FIG. 5A) Schematic representation of cross-chain EpiScore, describing i, j, in chains B and C, respectively, and its impact in receptor-binding domain (RBD) binding position k in the open RBD conformer chain A. (FIG. 5B) Colors indicate EpiScore values for given mutation pairs, averaged over hACE2 binding sites. Cross-chain residue pairs in the upper right tend to be highly epistatic, similar to pairwise second-order interaction coefficients from Moulana et al., 2022.

FIGS. 6A-6C show EpiScores of variants of concern (VOC) mutations. (FIG. 6A) Here, i=low evolutionary probability (EP) Delta mutation sites (magenta), low EP Omicron mutation sites (cyan), and a random selection of sites (gray), j=low EP sites in the Wuhan variant, and k=the binding interface of the open chain. EpiScores using sites of either variant (Delta: M=0.70, SD=0.50, Omicron M=0.86, SD=0.46) are significantly different (p<0.001) from a set of EpiScores using random sites (M=0.84, SD=0.41), but the distribution for Delta variants differs much more from the other two. (FIG. 6B) EpiScores with i=characteristic mutation sites, j=low evolutionary probability (EP) sites, and k=the binding interface of the open chain. EpiScores using variant sites are significantly different (p<0.001) from a set of EpiScores using random sites. (FIG. 6C) EpiScores with i=characteristic mutation sites within the NTD, j=low evolutionary probability (EP) sites, and k=the binding interface of the open chain. NTD mutation sites result in markedly lower EpiScores compared to elsewhere (p<0.001).

FIGS. 7A-7B show EpiScores with candidate adaptive polymorphism (CAP) site 486. (FIG. 7A) Here, i=low evolutionary probability (EP) Delta mutation sites (magenta), low EP Omicron mutation sites (cyan), and a random selection of sites (gray), j=site 486, and k=the binding interface of the open chain. CAP and hACE2 and antibody binding site 486 displays epistasis with almost all XBB 1.5 variant sites at almost every hACE2 binding site (M=1.40, SD=0.46) and presents a significantly different profile from other variant sites (p<0.01). (FIG. 7B) EpiScores with i=characteristic mutation sites, j=site 486, and k=the binding interface of the open chain. CAP and hACE2 and antibody binding site 486 displays epistasis with almost all XBB 1.5 variant sites at almost every hACE2 binding site. EpiScores using variant sites are significantly different (p<0.001) from a set of EpiScores using random sites.

FIGS. 8A-8E show the % DFI calculations for Omicron, XBB, and XBB 1.5. (FIG. 8A) % DFI profile of the variants are plotted in the same panel. The gray shaded areas and dashed lines indicate the ACE2 binding regions, whereas the red dashed lines show the antibody binding residues. (FIG. 8B) The sum of % DFI values of RBD-ACE2 interface residues. The trend of total % DFI with the log of K_{d} values overlaps with the one seen with the experiments (r=0.97). (FIG. 8C) The receptor-binding domain (RBD) antibody binding residues are used to calculate the sum of % DFI. The ranking captured with the total % DFI agrees with the log of IC50 values from the experiments. (FIG. 8D) Three critical mutations that emerged quickly and were frequently observed in other dominant variants are S477N, T478K, and N501Y EpiScores of sites 477, 478, and 501 with one another are shown (with k=the binding interface of the open chain). These residues are highly epistatic, producing higher responses than expected when perturbed together. (FIG. 8E) The difference in the dynamic flexibility profiles between the single mutants and the most common variants for the hACE2 binding residues of the receptor-binding domain (RBD). The dynamic flexibility index (DFI) profiles exhibit significantly different flexibility in each background variant, highlighting the critical non-additive interactions of the other mutation in the given background variant. Thus, these three binding affinity-impacting mutations do not contribute only their own effects to the binding interface, there are epistatic interactions with the other mutations in variants of concern (VOCs) that shape the dynamics of the binding interface to modulate binding.

FIG. 9 shows an example process of characterizing a protein in accordance with some embodiments of the disclosure.

FIG. 10 shows an example system in accordance with some embodiments of the disclosure.

### DETAILED DESCRIPTION

In accordance with some embodiments of the disclosed subject matter, mechanisms (which can include, for example, systems and methods) for characterizing a protein are provided. Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

The role of protein dynamics in viral evolution remains largely unexplored. To address this, we developed EpiScore, a computational tool that quantifies the epistatic interactions between pairs of mutations in the spike protein through a dynamic coupling index (DCI) approach. By analyzing the dynamic interplay of mutations, EpiScore can reveal novel insights into the evolutionary trajectory of the virus, potentially leading to more effective antiviral strategies that target the dynamic nature of the spike protein.

EpiScore is a groundbreaking computational tool specifically developed to forecast and characterize epistatic interactions within proteins (for example the SARS-CoV-2 spike). This innovative tool examines the dynamic interaction of residues within the protein, offering valuable insights into the non-additive effects of mutations on protein function. EpiScore plays a vital role in comprehending the intricate interplay of mutations in emerging variants of concern (VOCs), assisting in the prediction of future variants and the development of effective antiviral therapies. By offering a more in-depth understanding of the interactions between mutations, EpiScore has considerable commercial potential in drug discovery, diagnostic development, and vaccine design, ultimately aiding in the fight against COVID-19.

The integration of evolutionary predictions and protein dynamics analysis to provide a new, comprehensive understanding of molecular adaptations in the SARS-CoV-2 spike protein. By employing unique computational tools like DCI_{asym} and EpiScore, we can capture the asymmetric nature of dynamic coupling and the epistatic interactions between mutations, offering mechanistic insights into the allosteric control of the receptor-binding domain (RBD) and the functional consequences of mutations in various Variants of Concern (VOCs). This knowledge can be leveraged to inform the development of more effective treatments and vaccines against a virus (e.g., SARS-CoV-2 and its emerging variants).

Exemplary methods and systems disclosed herein employ cutting-edge computational protein dynamics analysis to unveil the molecular mechanisms underlying adaptations in the SARS-CoV-2 spike protein. The process may involve the following elements:

1. Protein Dynamics Analysis: A suite of computational tools, including dynamic flexibility index (DFI), dynamic coupling index (DCI), and novel EpiScore, are utilized to assess the effects of CAPs on protein dynamics. DFI measures the flexibility of individual residues, while DCI measures the strength of dynamic coupling between residues. EpiScore examines the epistatic interactions between pairs of mutations to determine if their effects are additive or non-additive.

2. Epistatic Interactions: EpiScore analysis reveals the complex relationships between CAPs and mutations that are characteristic of different VOCs, such as Delta and Omicron. The method demonstrates that the non-additive interactions between these mutations can significantly impact the functional behavior of the spike protein.

3. Mechanistic Insights: Molecular Dynamics Simulations are performed to analyze protein dynamics and reveal how CAPs modulate the spike protein's dynamics, particularly at functionally critical sites such as the receptor-binding domain (RBD) and the Furin cleavage site. The method unveils the allosteric control exerted by CAPs on the dynamics of the RBD, influencing its accessibility for binding to the human ACE2 receptor.

The systems and methods described herein offer numerous advantages compared to conventional methods for investigating viral evolution. In some embodiments, the systems and methods described herein not only identify individual mutations but also delves deeper into the molecular mechanisms that drive viral adaptations. In further embodiments, the systems and methods described herein provide the analysis of protein dynamics which allow for the prediction of functional outcomes of future mutations and the possible emergence of new Variants of Concern (VOCs). Additionally, the insights gleaned from the systems and methods described herein can guide the development of efficient antiviral therapies that focus on targeting the dynamic aspects of the spike protein.

The systems and methods described herein hold substantial commercial prospects in several key sectors, including but not limited to drug discovery, diagnostic development, and vaccine design. In some embodiments, the systems and methods described herein may identify undiscovered drug targets that have the ability to regulate a spike protein's dynamics. In further embodiments, the ability to regulate a spike protein's dynamics may inhibit viral infiltration and/or replication. In even further embodiments, the ability to regulate a spike protein's dynamics may offer a treatment for a disease. In some embodiments, the systems and methods described herein may integrate analysis of a protein dynamic into a diagnostic tool. In further embodiments, integration of the analysis of a protein dynamic into the tool may facilitate detection and/or characterization of an emerging Variant of Concern (VOC). In further embodiments, integration of the analysis of a protein dynamic into the tool may provide the ability to identify and/or monitor a new viral strain. In some embodiments, the systems and methods described herein may be applied to the development of a vaccine. In further embodiments, the developed vaccine may provoke a more comprehensive immune response against a virus and/or a range of its variants (e.g., SARS-CoV-2). In some embodiments, the developed vaccine may improve vaccine efficacy.

In one aspect, the disclosure provides methods and systems of characterizing a protein. The method may include using a computer system to: access simulated protein structure data with a computer system, in which the simulated protein structure data indicate a structure of the protein; select a plurality of residues in the protein and quantifying a plurality of dynamics metrics corresponding to each residue in the plurality of residues, in which the dynamics metrics comprise a metric of epistasis (EpiScore); identify at least one residue of interest based on the plurality of dynamics metrics; and characterize the protein and the at least one residue of interest based on the plurality of dynamics metrics. In some embodiments, the method further includes generating a report of the characterized protein and providing the report to a user.

In some embodiments, “a plurality of residues” includes many residues in the protein that are known to be, or are suspected to be, functionally important sites. These sites may be active sites, catalytic sites, allosteric sites, etc. In some embodiments, the plurality of residues may include residues that are known to be located at binding interfaces of a protein. In some embodiments, a plurality of residues may include one or more residue that is not known or suspected to be a functionally important site; such a residue may later be proved to be functionally important.

In some embodiments, the metrics of epistasis may be referred to as an “EpiScore.” An EpiScore quantifies the epistasis of a residue of interest at a given position when force is applied at a pair of residues. The position of each residue in the pair of residues is not the position of the residue of interest (e.g., the residue of interest is at position k, the pair of residues are located at position i and j). In some embodiments, an EpiScore of 1 indicates perfect coupling additivity, and deviations from 1 represent non-additive behavior between position pairs and functionally important sites.

In some embodiments, further dynamics metrics may include, but are not limited to, dynamic flexibility index, dynamic coupling index, or asymmetric dynamic coupling index.

A “residue of interest” may be identified based on the dynamics metrics, particularly the EpiScore. The residue of interest may be indicated to be a potential allosteric site.

In some embodiments, the method may further include accessing evolutionary information regarding the protein and calculating an evolutionary probability (EP) score for each residue in the plurality of residues. This may be used to identifying candidate adaptive polymorphisms (CAPs) in the protein.

In some embodiments, the method may further include comparing the residues of interest with CAPs identified using evolutionary data. This may help further determine which residues are likely to be mutated, and which residues are likely to play a role in virus function.

In some embodiments, the protein of interest is a viral protein (e.g., a protein from a virus). The virus may include rhonovirus, influenza virus, varicella, coronavirus (including SARS-CoV2), respiratory syncytial virus, human metapneumovirus, adenovirus, parainfluenza virus, norovirus, rotavirus, mpox, Ebola, measles, rubella, or mumps.

In some embodiments, the methods and systems described herein may be used to characterize and predict viral characteristics and behavior. Functional behaviors of a virus include, but are not limited to, predicted antibody binding, pathogenicity, effective immune escape, and transmission rate.

In some embodiments, the methods and systems may be used to compare viral variants. In some embodiments, corresponding proteins from multiple viral variants may be analyzed and characterized using the methods and systems described herein. The epistasis metrics and/or CAPs can be compared across the viral variants, and then linked to viral characteristics and behavior. The viral variants may include different known strains of the virus, or a known strain and a new, uncharacterized strain.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 'B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, or example, by a mutated residue. A protein, peptide, or polypeptide may also be a single molecule or may be a multi-molecular complex. A protein, peptide, or polypeptide may also be a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain.

A “site” or “residue” may refer to an amino acid at a specific position in the protein. The terms may be used to refer to the position, or the specific amino acid at said position. The terms site and residue are used interchangeably throughout the specification.

### EXAMPLES

The following examples are meant to be illustrative and should not be seen as limiting.

### Example 1: Some Mechanistic Underpinnings of Molecular Adaptations of SARS-COV-2 Spike Protein by Integrating Candidate Adaptive Polymorphisms with Protein Dynamics

We integrate evolutionary predictions based on the neutral theory of molecular evolution with protein dynamics to generate mechanistic insight into the molecular adaptations of the SARS-COV-2 spike (S) protein. With this approach, we first identified candidate adaptive polymorphisms (CAPs) of the SARS-CoV-2 S protein and assessed the impact of these CAPs through dynamics analysis. Not only have we found that CAPs frequently overlap with well-known functional sites, but also, using several different dynamics-based metrics, we reveal the critical allosteric interplay between SARS-CoV-2 CAPs and the S protein binding sites with the human ACE2 (hACE2) protein. CAPs interact far differently with the hACE2 binding site residues in the open conformation of the S protein compared to the closed form. In particular, the CAP sites control the dynamics of binding residues in the open state, suggesting an allosteric control of hACE2 binding. We also explored the characteristic mutations of different SARS-CoV-2 strains to find dynamic hallmarks and potential effects of future mutations. Our analyses reveal that Delta strain-specific variants have non-additive (i.e., epistatic) interactions with CAP sites, whereas the less pathogenic Omicron strains have mostly additive mutations. Finally, our dynamics-based analysis suggests that the novel mutations observed in the Omicron strain epistatically interact with the CAP sites to help escape antibody binding.

Since 2019, the evolution of SARS-CoV-2 in humans has been characterized by the spread of mutations, many notably found within the spike (S) glycoprotein. The S protein is directly related to the human immune response to COVID-19 and, as such, has been one of the most studied and targeted proteins in SARS-CoV-2 research (Shang et al., 2020; Harvey et al., 2021; Jackson et al., 2022; Carabelli et al., 2023; Markov et al., 2023). Subsequently, research into the biophysical properties and mutational patterns associated with S protein evolution not only remains critical to understanding the pandemic but also emerges as a useful system to understand the mechanics of molecular adaptation within viruses.

For successful infection of a human host, the S glycoprotein of SARS-CoV-2 binds to the human ACE2 (hACE2) receptor through its receptor-binding domain (RBD). Evidence indicates that fine-tuning S protein interactions with hACE2 significantly affects viral reproduction (Rehman et al., 2020; Saputri et al., 2020; Rochman et al., 2021). Previous evolutionary studies show a complex network of interactions among mutated residues (Changeux and Edelstein, 2005; Doshi et al., 2016; O'Rourke et al., 2016; Mishra and Jernigan, 2018). Therefore, there has been a vast effort to uncover which mutations are important steps of adaptation for the S protein (Cagliani et al., 2020; Damas et al., 2020; Singh and Yi, 2021; Kistler et al., 2022; Maher et al., 2022; Neher, 2022). In particular, a significant aspect of many such studies was a focus on understanding adaptive mutations of SARS-CoV-2 that contributed to the leap to human hosts (Cagliani et al., 2020; Damas et al., 2020; Singh and Yi, 2021; Starr et al., 2022c). This is because SARS-CoV-2 has continuously mutated since its early detection (Kistler et al., 2022), causing the emergence of CDC-designated ‘variants of concern’ (VOCs) that may be driven by an accelerated substitution rate (Tay et al., 2022).

Predicting how new mutations impact the biophysical properties of the S protein remains a challenge, let alone explaining their complex interactions with one another and how they might affect hACE2 binding; many factors affect hACE2 interactions. Binding affinity with hACE2 can be enhanced directly through stronger receptor interactions or mediated through changes in RBD opening (Teruel et al., 2021b; Zhang et al., 2021; Diaz-Salinas et al., 2022). The RBD exhibits both ‘closed’ and ‘open’ conformational states. In the closed state, the RBD is shielded from receptor binding. In the open state, the RBD is accessible for hACE2 binding (Kirchdoerfer et al., 2016; Gur et al., 2020; Henderson et al., 2020; Hoffmann et al., 2020). While some mutations may affect the transition between these states (Henderson et al., 2020; Yurkovetskiy et al., 2020; Gobeil et al., 2021a; Sztain et al., 2021; Zhang et al., 2021; Shoemark et al., 2022), other mutations may allosterically regulate RBD openings through Furin cleavage site (residue ID range: 681-695) interactions to regulate hACE2 binding (Deng et al., 2021; Gobeil et al., 2021b; Khan et al., 2021; Laiton-Donato et al., 2021).

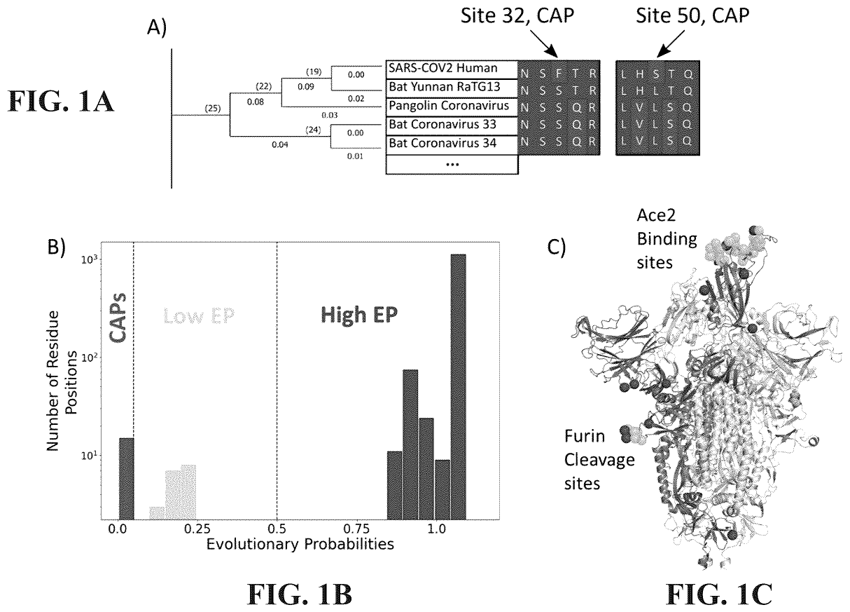

Moreover, as new mutations accumulate, culminating in the emergence of a new VOC, these mutations must occur on varied sequence backgrounds containing neutral, nearly-neutral, and adaptive mutations. While many studies have explored the impacts of individual mutations, VOCs result in a substantial difference in protein function compared to their individual effects (Moulana et al., 2022; Starr et al., 2022c; Moulana et al., 2023; Witte et al., 2023). Here we integrate an evolutionary approach with protein dynamics analysis to address the fundamental mechanisms of mutations dictating VOCs and the impact of their epistatic interaction on the function of the S protein. Many earlier studies have combined phylogeny and evolutionary theory to identify adaptive mutations and analyze how the viral sequence has changed over time (Frost et al., 2018; Boni et al., 2020; Cagliani et al., 2020; Damas et al., 2020; Tang et al., 2020). Similarly, we first use a well-established evolutionary probability (EP) approach (Liu et al., 2016) that utilizes phylogenetic trees in combination with the neutral theory of molecular evolution to determine candidate adaptive polymorphisms (CAPs) using the early Wuhan sequence as a variant. CAPs are substitutions in SARS-CoV-2 that are rarely observed in other closely related sequences (FIG. 1A), which implies a degree of functional importance and makes them candidates for adaptation (Liu et al., 2016). Adaptation in this case means a virus which can successfully infect human hosts. As CAPs are unexpected polymorphisms under neutral theory, their existence implies a non-neutral effect. This can come in the form of functional changes (Liu et al., 2016) or compensation for functional changes (Ose et al., 2022b). Therefore, we suspect that these CAPs may be partially responsible for the functional change allowing the infection of human hosts. In support of this method, we find an overlap between our list of sites containing CAPs and putative adaptive sites identified by others (Cagliani et al., 2020; Singh and Yi, 2021; Kistler et al., 2022; Starr et al., 2022b). Second, we use a suite of computational tools to analyze how CAPs that arose in the early and late phases of the COVID-19 pandemic modulate the dynamics of the S protein. We also explore the complex interactions between these sets of CAPs to gain mechanistic insight into the behavior of molecular adaptation involving the S protein. In particular, we focused on how mutations modulate protein dynamics as we and others have previously found that rather than changing a protein's structure solely, mutations modulate conformational dynamics, leading to changes in biophysical properties such as stability, flexibility, and allosteric dynamic coupling, any of which may affect protein binding (Swint-Kruse et al., 1998; Keskin et al., 2000; Bhabha et al., 2013; Nussinov and Tsai, 2013; Campbell et al., 2016; Ma and Nussinov, 2016; Saavedra et al., 2018; Kuzmanic et al., 2020).

With this evolutionary-dynamics unified approach, we aim to answer the following questions about VOCs: Are all the mutations in VOCs adaptive in nature? Are they coupled to one another or provide some measure of biophysical, dynamical, or mechanical epistasis? While many of these mutations are found within the RBD, numerous others are located distal to this region; hence, we aim to investigate the functional roles of distal mutations, particularly from a protein dynamics perspective. Our integrated analysis revealed that protein dynamics play a significant role in the evolution of the S protein. The flexibility of sites within the S protein shows a strong, direct correlation with substitution rate, and newly evolving CAPS are mostly additive mutations that modulate the dynamics of the hACE2 binding site. Yet other CAPs, 346R, 486F, and 498Q, show highly epistatic (i.e., non-additive) modulation of the hACE2 binding site and provide immune escape benefits.

SARS-CoV-2 is part of a family of coronaviruses, many of which infect mainly animals and are less capable of infecting humans (Dicken et al., 2021). Therefore, to identify the most likely mutations responsible for the infection of human hosts (i.e., putative adaptive mutations for humans), we estimated the (neutral) EP scores of mutations found within the S protein (Liu et al., 2016). EP scores of the amino acid variants of the S protein were obtained using a maximum likelihood phylogeny (Kumar et al., 2018) built from 19 orthologous coronavirus sequences. Sequences were selected by examining available non-human sequences with a sequence identity of 70% or above to the human SARS CoV-2's S protein sequence. This cutoff allows for divergence over evolutionary history such that each amino acid position had ample time to experience purifying selection, whilst limiting ourselves to closely related coronaviruses. (FIG. 1A). The likelihood of finding a particular amino acid in the sequence is then determined using a Bayesian framework, with calculations carried out by MEGA X software (Kumar et al., 2018). As apparent in the name, EP scores obtained for the amino acids in the sequence provide information regarding the likelihood of finding them at their position, given the history of the sequence. Amino acid residues receiving low EP scores (<0.05) at a position are less likely to be found in a given position within the sequence because they are non-neutral. Generally, positions with low EP amino acids are far less common than those containing mutations with high EP, a trend also realized in the CoV-2 S protein (FIG. 1B).

Of particular interest is an observed evolutionary change where an amino acid with high EP is replaced by an amino acid residue with low EP. While amino acids with low EP should be harmful or deleterious to viral fitness due to functional disruption or change, fixation of a low EP amino acid at a position suggests an underlying mechanism for natural selection to operate. These fixed, low EP mutations are called CAPs as they are predicted to alter protein function, and adaptive pressures may drive their prevalence (Kumar and Patel, 2018). Indeed, there is an overlap between these CAPs and the mutations suggested by other methods to be adaptive for the S protein (Cagliani et al., 2020; Singh and Yi, 2021; Kistler et al., 2022; Starr et al., 2022b). Within these studies, sites 478, 486, 498, and 681 have been implicated in SARS-CoV-2 evolution, leaving the remaining 11 CAPs as undiscovered candidate sites for adaptation.

Interestingly, most of the CAP residues are at functionally critical sites, including the RBD and the Furin cleavage site (FIG. 1C). As mentioned earlier, the RBD plays a key role in initiating the infection of a healthy cell by binding with the host organism's ACE2 protein. Before ACE2 binding, one chain of the homotrimer comprising the S protein must open to expose the RBD (Kirchdoerfer et al., 2016; Henderson et al., 2020; Hoffmann et al., 2020; Sztain et al., 2021). The Furin cleavage site plays a key role in the opening process as the binding of host cell protease Furin aids in the cleavage of the S protein into two domains: S1 and S2 (Wrobel et al., 2020: 13). Another host cell protease, TMPRSS2, facilitates viral attachment to the surface of target cells upon binding either to sites Arg815/Ser816, or Arg685/Ser686 which overlap with the Furin cleavage site 676-689, further emphasizing the importance of this area (; Fraser et al., 2022). Similar cleavage sites have been found in related coronaviruses, including HKU1 and Middle East respiratory syndrome coronavirus (MERS-CoV), which infects humans (Chan et al., 2008: 1; Millet and Whittaker, 2014; Millet and Whittaker, 2015), and the acquisition of similar cleavage sites is associated with increased pathogenicity in other viruses such as the influenza virus (Steinhauer, 1999). Interestingly, however, CAPs do not display such an overwhelming tendency to occur at well-known critical sites within human proteins studied with similar methods (Ose et al., 2022b), yet mutations at those sites are associated with disease, indicating their critical role in inducing functional change. Therefore, the identified CAPs in the S protein, which are signs of recent evolution, can provide mechanistic insights regarding the molecular adaptation of the virus. In particular, we aimed to analyze how these CAP positions in the S protein modulate the interaction with hACE2 using our protein dynamics-based analysis (Gerek and Ozkan, 2011; Nevin Gerek et al., 2013; Larrimore et al., 2017; Kumar et al., 2015).

A mutation at a given amino acid position inevitably not only alters local interactions, but this change cascades through the residue-residue interaction network, which gives rise to a variation in native ensemble dynamics to modulate function (Dror et al., 2012; Labbadia and Morimoto, 2015; Sekhar and Kay, 2019; Campitelli et al., 2020.). Many groups have already examined the conformational dynamics of the S protein using normal mode analysis to explore mutation sites and interactions with different receptors (Zhou et al., 2020; Majumder et al., 2021; Teruel et al., 2021a; Verkhivker, 2022). However, our study will focus mainly on the role of CAPs, of which F486 and Q498 have already been identified through perturbation response scanning (PRS) as potential allosteric sites (Verkhivker, 2022). Thus, we analyze the internal dynamics of the system to understand the functional role of CAPs in S proteins. This analysis allows us to gain a mechanistic understanding of the relationship between CAP mutations and biophysical outcomes (Teruel et al., 2021b). First, we implement the dynamic coupling index (DCI) approach to study long-distance coupling between the CAPs and the hACE2 binding sites emerging from the 3D network of interactions across the S protein system. DCI calculation combines PRS and linear response theory (LRT) to capture the strength of a displacement response for position i upon perturbation of position j, relative to the average fluctuation response of position i to all other positions in the protein. It represents the strength of dynamic coupling between positions i and j upon perturbation to j (Larrimore et al., 2017; Kumar et al., 2015).

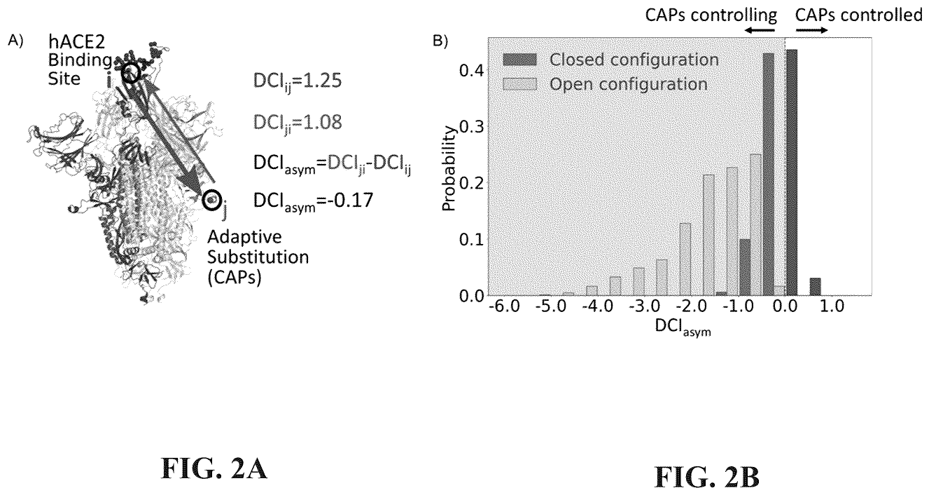

Further, asymmetry can be captured in the DCI values as dynamic coupling is not necessarily symmetric due to an anisotropic interaction network. That is, each amino acid has a set of positions to which it is highly coupled, and this anisotropy in connections gives rise to unique differences in coupling between a given i, j pair of amino acids which do not have direct interactions. By calculating the coupling of the hACE2 binding interface in the RBD with respect to the CAP residue positions and vice versa, we can generate DCI_{asym} (FIG. 2A) as the difference between the normalized displacement response of position j upon a perturbation to position i (DCIᵢⱼ) and the normalized displacement response of position i upon a perturbation to position j (DCIⱼᵢ) (see Methods). If the DCI_{asym} values significantly differ from zero, it shows asymmetry in coupling and presents a cause-effect relationship between the i, j pair in terms of force/signal propagation. This metric has been used previously in a variety of systems to analyze the unique behavior of positions within a protein and a given position's propensity to effect biophysical changes upon mutation, particularly at long distances (Modi and Ozkan, 2018; Campitelli and Ozkan, 2020; Kolbaba-Kartchner et al., 2021; Ose et al., 2022a; Kazan et al., 2023; Campitelli et al., 2021).

Recent work from our group has shown an enhancement in cross-chain communication within the main protease of SARS COV-2 compared to SARS COV-1 (Campitelli et al., 2022). Furthermore, previous studies have shown that allosteric inter-chain communication is important to S protein function (Zhou et al., 2020; Spinello et al., 2021; Tan et al., 2022; Xue et al., 2022). In support of these findings, we observe through DCI_{asym} that when the S protein is in its pre-fusion conformation with one chain open, the CAPs in the closed chains have negative coupling asymmetry with respect to the hACE2 binding site interface in the RBD-open chain. This indicates an allosteric control where the hACE2 binding site is dominated by the dynamics of the CAPs in closed chains (FIG. 2B, yellow bars). As this open-state RBD is critical for the viral infection of host cells (Kirchdoerfer et al., 2016), our results suggest that this type of closed-to-open cross-chain interaction is important for viral proliferation. Our prior studies on DCI_{asym} show a similar trend in lactose inhibitor (LacI), a protein with a functional role in gene expression through binding DNA. The allosteric mutations (i.e., mutations on the sites that are far from the DNA binding sites) that alter DNA binding affinity not only exhibited unique asymmetry profiles with the DNA binding sites of LacI, but also regulated the dynamics of these binding sites (Campitelli et al., 2021).

Similarly, it is possible that mutations to such residue positions within the S protein can be used to regulate the dynamics of the hACE2 bindings sites of the open RBD state. We, therefore, propose that the residue positions with CAP substitutions hold the potential for mutations in the spike sequence which can alter the opening and closing dynamics of the RBD. This hypothesis is further supported by many mutations already observed at these residue positions which alter the infection rate (Brister et al., 2015). Interestingly, residues responsible for extremely low asymmetry values (<−4) lie overwhelmingly in the region 476-486. These same residues were suggested to stabilize S protein dynamics and prime it for host Furin proteolysis (Raghuvamsi et al., 2021).

Moreover, as a control, we performed the same analysis on the S protein with the RBDs of all chains in the closed configuration. In this case, we observed that the DCI_{asym} of the CAPs residue positions with respect to the hACE2 interface in the other chains yields a largely symmetric distribution about 0 (FIG. 2B, green bars). This verifies that the asymmetry in the coupling of CAPs with the exposed binding site interface in pre-fusion configuration results from one of the RBDs opening up and further suggests the allosteric role played by CAPs in locking the S protein in the RBD open state.

CAPs represent important S protein amino acid changes between related coronaviruses across multiple species and the Wuhan-Hu-1 reference sequence (MN908947). Since SARS-CoV-2 first spread to humans, it has continued to mutate and evolve rapidly, particularly regarding the S protein (Amicone et al., 2022; Liu et al., 2022; Tay et al., 2022). Just as mutations leading to the Wuhan strain caused an increase in binding affinity to hACE2, continued evolution in human hosts has resulted in further altered binding affinities as well as different phenotypic outcomes for those infected (Ali et al., 2021; Barton et al., 2021; Ozono et al., 2021).

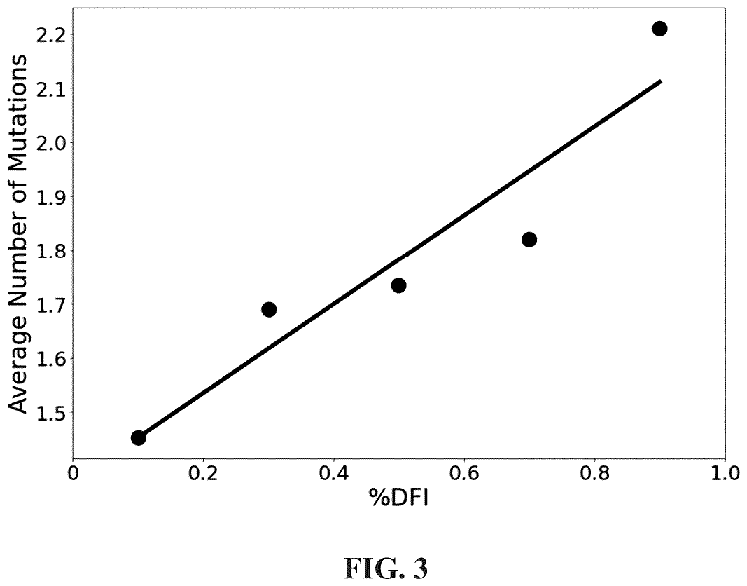

We explore whether protein dynamics has played a role in the selection of mutational sites during the evolution of the S protein since 2019. Our previous work has indicated that the rate of evolution per positional site exhibits a positive correlation with positional flexibility; generally, positions that exhibit higher flexibility are also sites that experience a higher number of amino acid substitutions (Liu and Bahar, 2012; Maguid et al., 2008; Maguid et al., 2006; Mikulska-Ruminska et al., 2019; Nevin Gerek et al., 2013). To confirm these findings for the evolution of the S protein using the sequenced variants of infected humans, we examine the flexibility of the S protein, an analysis conducted in other studies which resulted in several important findings (Nguyen et al., 2020; Socher et al., 2021; Teruel et al., 2021b; Pipit6 et al., 2022; Verkhivker, 2022; Abduljalil et al., 2023). For example, Teruel et al., 2021a used their Elastic Network Contact Model to find how certain highly observed mutations make the open state more rigid and the closed state more flexible. For our own flexibility analysis, we measure the site-specific amino acid flexibility using the dynamic flexibility index (DFI). Using the same mathematical foundation as DCI, DFI evaluates each position's displacement response to random force perturbations at other locations in the protein (Gerek and Ozkan, 2011; Nevin Gerek et al., 2013), and it can be considered a measure of a given position's ability to explore its local conformational space. We found that the COVID-19 S protein shows the expected high correlation between the occurrence of mutations and site flexibility (FIG. 3) when we compare % DFI (DFI ranked by percentile) to the average number of variants per position found within a given % DFI bin. Previous studies have indicated that rigid residues are critical for functional dynamics, thus more likely to impact function if mutated and, generally, can lead to a loss of function and thus more conserved (Kim et al., 2015; Butler et al., 2018; Modi et al., 2021b; Modi et al., 2021a; Kazan et al., 2022; Ose et al., 2022a; Stevens et al., 2022; Kumar et al., 2015). This analysis also agrees with these previous studies and highlights the power of negative selection, in line with the neutral theory of molecular evolution, stating that deleterious mutations (i.e., those on the rigid positions) should be eliminated and therefore not observed (Kimura, 1983).

Continued mutations within human hosts have resulted in a multitude of variants. Indeed, by fitting various molecular clock models to genome sequence data, VOC emergence is punctuated by an episodic period of rapid evolution, with a substitution rate of up to fourfold greater than the background substitution rate (Kumar et al., 2021; Tay et al., 2022). With such an aggressive evolutionary rate, we are finding VOCs to consist of a number of different characteristic mutations, almost all of which are CAPs. Sequence differences between the various VOCs used in this article can be found in Table 1 and Table 2.

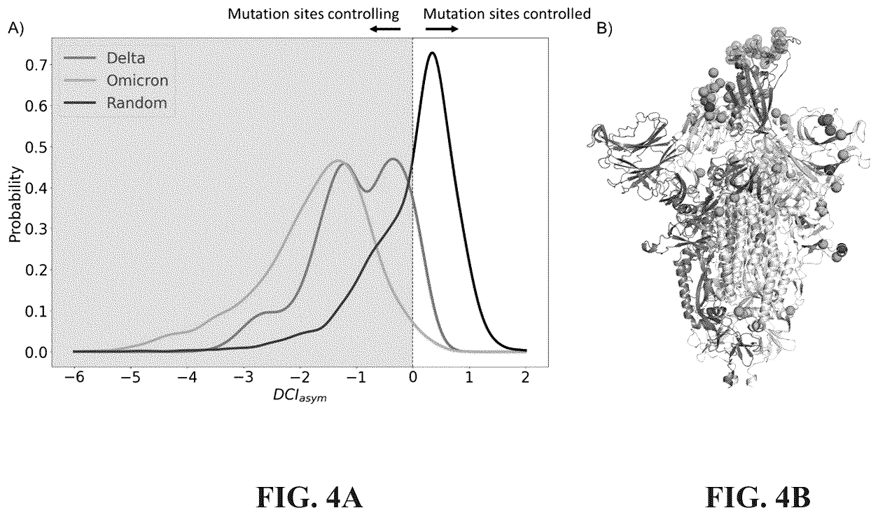

To explore the dynamic effects of the evolution of the spike in humans, we examine asymmetry with these new potentially adaptive sites, namely the low EP (CAP) characteristic mutation sites observed in the Delta variant, the widely dominant variant from December 2021 to January 2022 (Thye et al., 2021), and the Omicron variant, a highly transmissible variant whose lineages have remained dominant since January 2022 (Kim et al., 2021; FIGS. 4A-4B). This analysis revealed a mechanism similar to that for the CAPs in the reference protein (FIGS. 2A-2B) as the open-chain binding interface is also allosterically controlled by these potentially new adaptive sites. Regarding this, we see that the asymmetry is much more pronounced in observed mutations of Omicron variants, suggesting that these new mutations have a stronger power in controlling the dynamics of open-chain hACE2 binding interface compared to those observed in Delta variants. We surmise that the difference in virulence and infection rates between Omicron and Delta (Earnest et al., 2022; Bager et al., 2021; Sheikh et al., 2021; Twohig et al., 2022; Houhamdi et al., 2022; Menni et al., 2022) might be due to these specific CAPs within each variant and their differences in allosterically controlling the dynamics of open RBD binding sites as also observed in the DCI_{asym} analysis of the Wuhan variant in FIGS. 2A-2B.

The fact that the identified CAPs in the reference protein and the more recently evolved CAPs of Delta and Omicron variants both show a high degree of control over the functional sites begs the question: what is the complex interaction between these previous and new CAP sites? Motivated by this concept, we explore the interplay of mutational pairs to understand the effects of the specific amino acid backgrounds associated with these two predominant variants. Some CAP sites in Delta and Omicron have already been considered adaptive (Kemp et al., 2021; Kistler et al., 2022; Maher et al., 2022; Neher, 2022).

It is well understood that the impact of even a single mutation to a protein sequence can sometimes dramatically alter the biophysical behavior of the system. However, the mechanistic impact of point mutations can only be fully understood when the sequence background upon which it is made is accounted for. This means that, in the case of strains with multiple mutations, the interplay between mutated positions will ultimately impact a protein as an aggregate behavior, where the presence of previous mutations may strongly (or weakly) influence some mutations. This concept of non-additivity is known as epistasis. In fact, studies of evolutionary pathways of mutations have suggested that a majority of the mutations have a second or a higher order epistasis among them (Bershtein et al., 2006). Nature exploits this higher order complex relationship between the mutations to evolve their function.

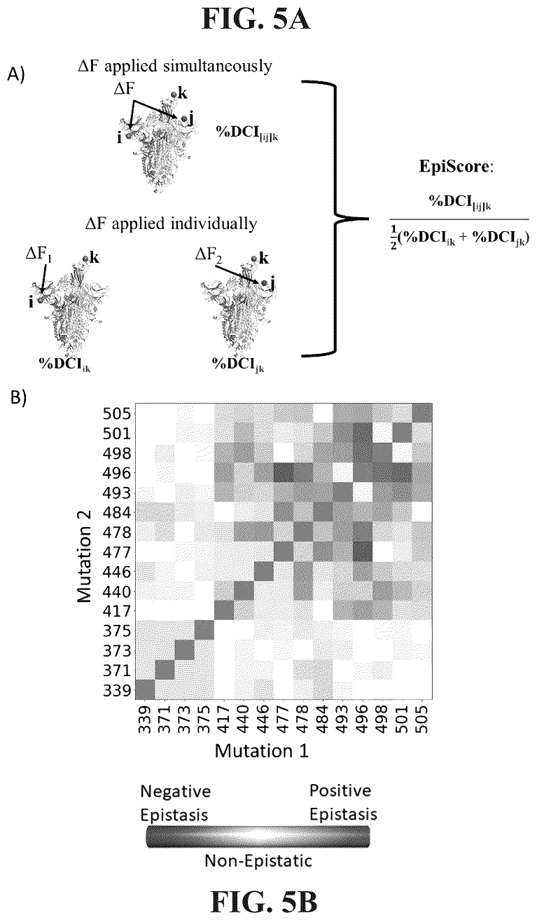

To computationally capture and interpret the pairwise effects of mutations, we have developed an in-house computational tool called EpiScore (FIG. 5A). Here, we evaluate how a given position pair i j may affect other critical positions k of the protein. EpiScore is the relative coupling strength to a position k when positions i and j are perturbed simultaneously compared to the average dynamic coupling strength of i to k and j to k. EpiScore has previously been used successfully to capture overarching trends in GB1 deep mutational scan data as well as specific instances of the development of antibiotic resistance in various enzymatic systems (Campitelli and Ozkan, 2020). An EpiScore of 1 indicates perfect coupling additivity, and deviations from this value represent non-additive behavior between position pairs and functionally important sites. Prior EpiScore work has shown a difference in EpiScore between the sites of compensatory and non-compensatory mutations, where both yield distributions with peaks around 1, but non-compensatory mutations show higher deviation in their EpiScore distribution (Ose et al., 2022b).

Many studies have confirmed epistasis between residues within the S protein (Moulana et al., 2022; Starr et al., 2022a; Moulana et al., 2023; Witte et al., 2023). These epistatic residues can have various effects on hACE2 or antibody binding. Within SARS-CoV-2, here we calculate the EpiScore (FIG. 5B) of a set of mutation pairs used by Moulana et al., 2022 and compare our results to quantified epistatic effects determined by the experimental hACE2 binding affinity of ‘first-order’ single mutation variants compared to ‘second-order’ mutation pairs. Our EpiScore results and the experimentally determined epistasis both captured highly epistatic behavior among residues 493, 496, 498, 501, and 505, as well as a lack of epistatic behavior for residues 339, 371, 373, and 375; however, EpiScore generally showed higher epistasis values than experiment for residues 417, 440, 446, 477, 478, and 484.

### EpiScore Highlights the Epistatic Relationship Between the Recent Adaptive Mutations in VOCs and the CAPs of the Wuhan Reference

Seeking further to understand the role of epistasis within S protein variants, we explored the possibility of epistatic relationships between the CAPs of the Wuhan variant and the new CAPs in VOCs. Thus, we computed the EpiScore of these CAP positions in the closed RBDs (i.e., chains B and C) with respect to functional hACE2 binding interface sites of the open RBD chain (chain A) (FIG. 5B) and obtained EpiScore distributions.

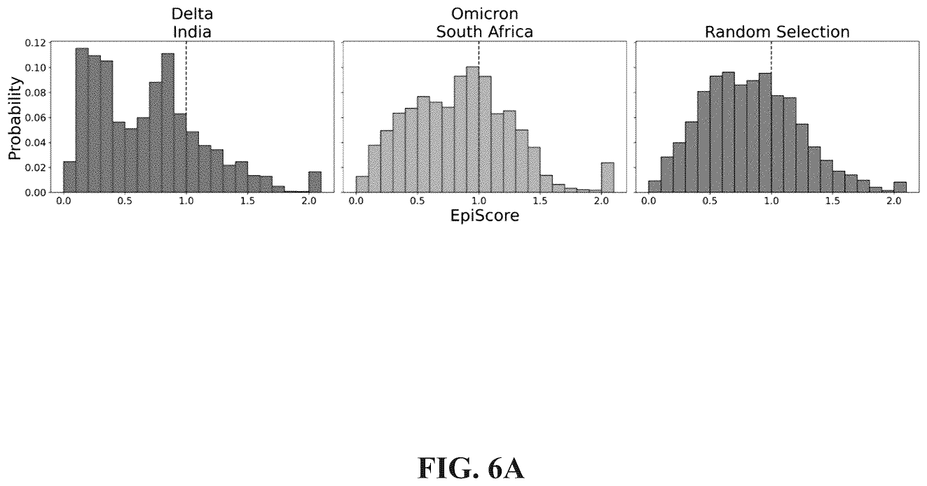

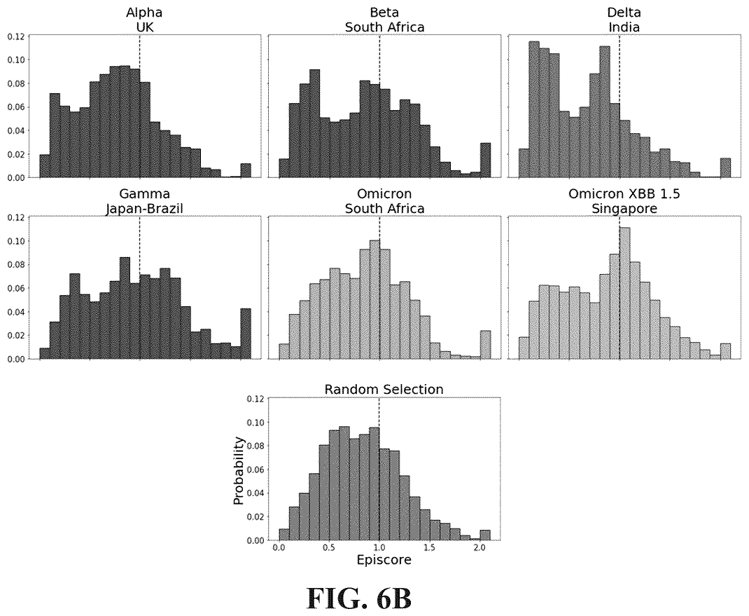

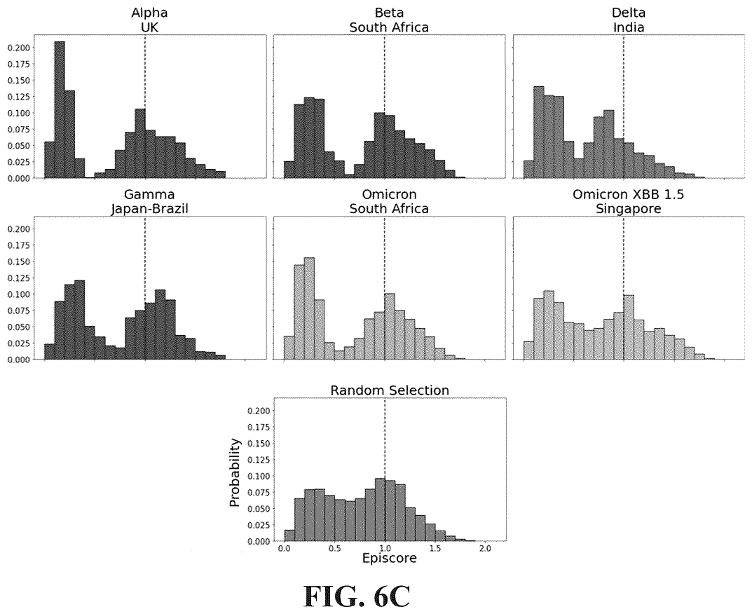

To contrast these variants, Omicron (FIG. 6A, cyan) shows a high proportion of additive mutations compared to the Delta variant (FIG. 6A, magenta), with a peak centered on 1. The comparatively more pathogenic Delta variant exhibited many non-additive mutations with EpiScores below one. This again suggests that the cross-communication between the open and closed chain of the S protein is important for regulating the function. Four out of seven low EP Delta mutation sites used in this analysis often resulted in EpiScores below 1. Each of those is found in the N-terminal domain (NTD) on or near the N3 loop and is implicated in antibody escape in recent studies (Chi et al., 2020; Weisblum et al., 2020; Harvey et al., 2021; Klinakis et al., 2021; Cantoni et al., 2022). The low EpiScores of NTD mutations suggest that they dampen the control of Wuhan variant CAPs over the hACE2 binding sites in addition to their effects on antibody binding. It is possible that what the Delta variant gained in transmission rate also came with being more harmfully pathogenic due in part to negatively epistatic interactions. In contrast, the mutations leading to the development of the Omicron strain were additive with respect to Wuhan variant CAPs, possibly leading to a lower pathogenicity and higher effective immune escape, resulting in an overall higher transmission rate. Another possible explanation for the higher transmission rate of Omicron comes from a different normal mode analysis study, which found that despite reduced binding affinity with hACE2, Omicron showed increased occupancy of the open state compared to the closed state, which would increase chances of interaction with hACE2 (Teruel et al., 2021b). It is worth noting that other variants contain NTD mutations which result in low EpiScores; however, the proportion of these mutations within the set is considerably less than in Delta (FIG. 6C).

One of the more notable features of generated EpiScore distributions is the presence of a tail of values upward of 2.0, indicating highly epistatic behavior. Interestingly, these tails are largely due to three different CAPs: 346R, 486F, and 498Q. Those residues are nearby one another within the protein structure and have been reported to play a role in antibody binding, either being known antibody binding sites (346R and 486F) or having received very high antibody accessibility scores (498Q) (Harvey et al., 2021; Raghuvamsi et al., 2021). These observed high EpiScore values also support other studies indicating the epistatic interactions between these CAPs and the mutations of the VOCs within the S protein are crucial for maintaining binding affinity of hACE2 whilst evading immunity (Hong et al., 2022; Starr et al., 2022b).

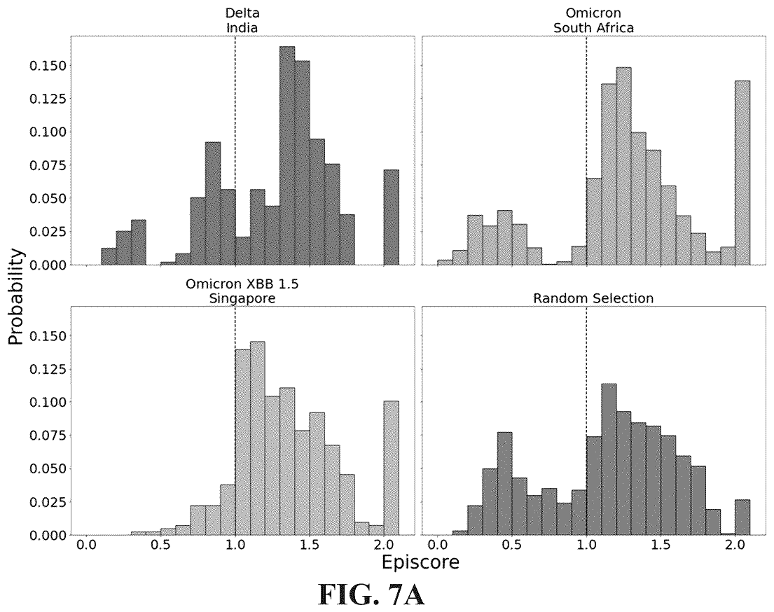

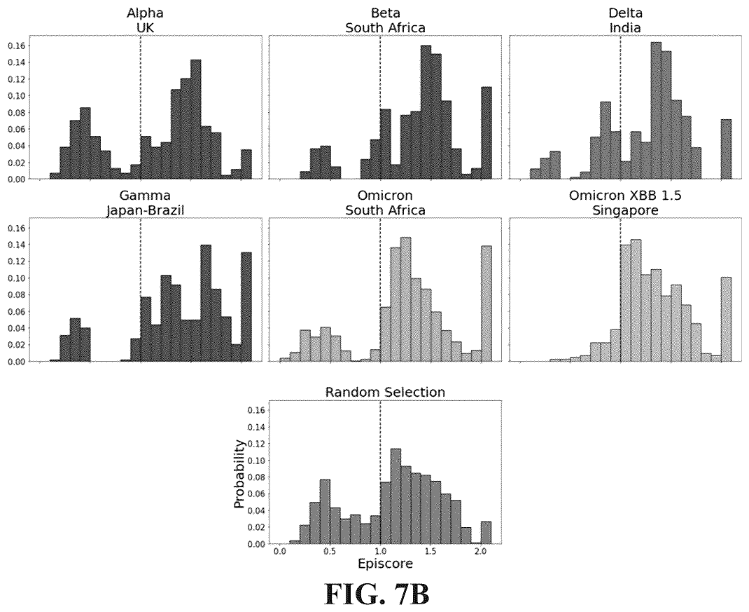

Inspection of EpiScores of Delta and Omicron potentially adaptive mutation sites with only CAP site 486F (a binding site for both hACE2 and antibodies) (Huang et al., 2020; Ali et al., 2021; Harvey et al., 2021; Raghuvamsi et al., 2021) shows highly epistatic interactions at other hACE2 binding sites (FIG. 6A). However, within a recent and rapidly spreading subvariant of Omicron, XBB 1.5, we see a mutation of S to P, a rare double nucleotide mutation, at site 486 (preceding Omicron variants included mutation F486S from the original CAP). This new variant has unprecedented immune escape capabilities, resisting neutralizing antibodies almost entirely (Qu et al., 2023). EpiScores of other XBB 1.5-specific mutation sites with 486P are almost entirely greater than 1, showing an even higher degree of epistasis with the binding sites of RBD (FIG. 7A). These results present a threefold importance for the S486P mutation: not only does this residue alter antibody (i.e., immune escape) and hACE2 binding by directly modifying a binding site, but it may also be responsible for modifying hACE2 binding via epistatic cooperation with other co-occurring mutations.

Experimental studies have tracked hACE2 binding for different variants since the virus first spread (Ali et al., 2021; Barton et al., 2021; Ozono et al., 2021; Wu et al., 2022). Within the Omicron variant, for example, characteristic mutations on the RBD are shown to increase the overall binding affinity of the virus to the ACE2 receptor, which is suspected to allow it to spread more easily (Kim et al., 2021). Furthermore, the new Omicron XBB and Omicron XBB 1.5 variants contain additional mutations in the RBD and antibody binding residues, which may further impact their dynamics and interactions with the host.

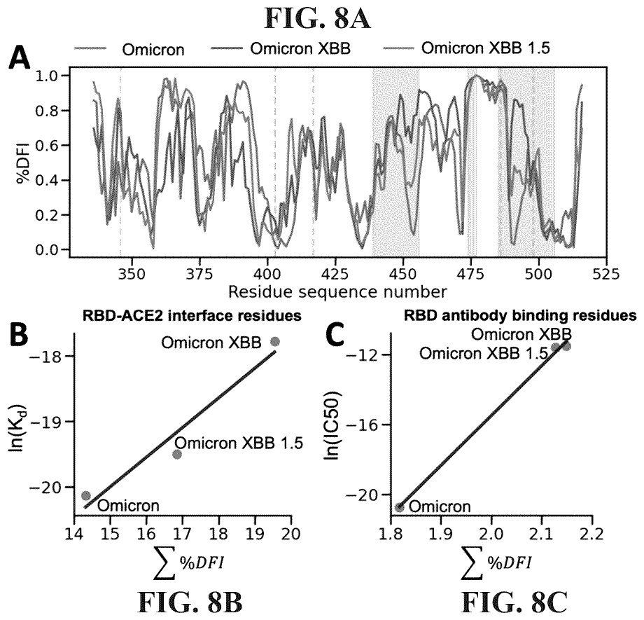

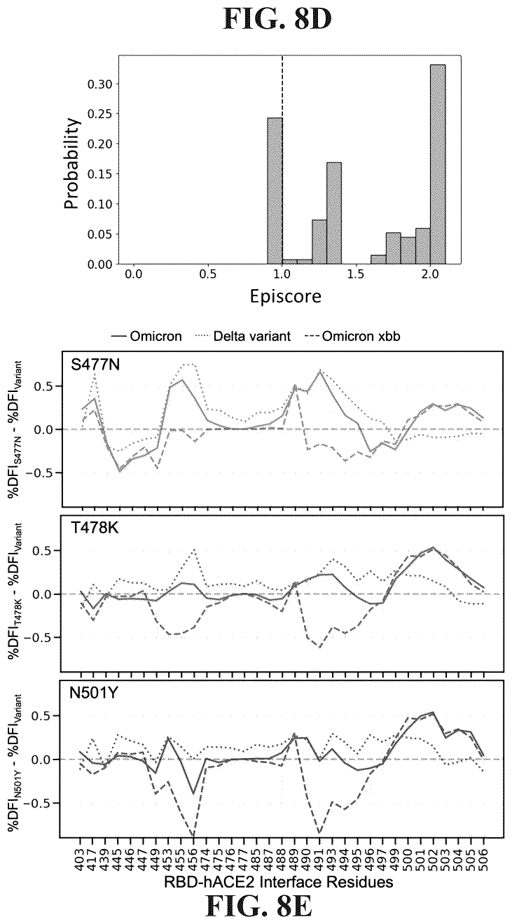

To gain deeper insights into the impact of dynamics on the binding affinity of hACE2 and antibodies with the recent Omicron XBB variants, we conducted molecular dynamics (MID) simulations. By analyzing the resulting trajectories, we investigated how these mutations influence the flexibility and rigidity of the RBD and antibody binding residues, consequently affecting their binding affinity and potential for immune evasion (FIGS. 8A-8E). To understand the overall flexibility changes, we measured the sum of DFI of the ACE2 binding residues, as well as the sum of DFI of the antibody binding residues, calculated from the MD trajectories and compared them with experimental viral binding (disassociation constants) and immunity evasion antibody IC50 values (Yue et al., 2023).

This investigation elucidated the impact of mutations in the RBD and antibody binding residues on the binding affinity of the S protein and immune evasion by modulating their flexibility and rigidity (FIG. 8A). The Omicron XBB variant exhibits heightened flexibility in hACE2 and antibody binding residues, reducing infectivity and enhancing immune evasion. Conversely, the Omicron XBB 1.5 variant induces distinct dynamics in these regions, rendering the RBD-ACE2 interface more rigid while increasing flexibility in antibody binding residues. These effects indicate that Omicron XBB 1.5 retains its antibody escape capabilities while regaining ACE2 binding affinity comparable to previous Omicron variants, in accordance with experimental findings (Yue et al., 2023). These findings suggest that mutations in the RBD and antibody binding residues can have complex effects on the dynamics of the protein and, ultimately, on the virus's ability to infect and evade the host immune system through an alteration of binding site dynamics. However, we do note that the effect of mutations will heavily depend on the genetic background in which they are mutated. The effects of a mutation on a Delta variant protein may differ entirely from the effects of a mutation on an Omicron variant protein (FIGS. 8D-8E).

We analyzed the evolutionary trajectory of the CoV-2 S protein in humans to understand the dynamic and epistatic interactions of the mutations defining specific VOCs. We first obtain the phylogenetic tree of the COV-2 S protein and identify the sites of certain recent mutations known as CAPs. CAPs are considered adaptive because mutations rarely tolerated in closely related sequences have suddenly become fixed, implying a degree of functional importance or evolution (Liu et al., 2016). In addition, our earlier work has shown that CAPS can also be compensatory; multiple CAPs may dynamically compensate for one another, changing the dynamic landscape and allowing for different mutations (Ose et al., 2022b). We then explored the mechanistic insights and epistatic relationship between the observed mutations in different VOCs and CAP sites, and, particularly, the relationship between CAP sites and the functionally critical RBD using our dynamic coupling analysis (Kumar et al., 2015).

We find a mechanistic pattern in the S protein evolution that is common amongst previously studied systems, where allosteric sites exert control over the dynamics of the binding sites, and mutations of these allosteric sites modulate function. Coupling our analysis with evolutionary theory showed that many of these allosteric sites regulating function of the S protein may have been subjected to adaptive evolution as observed in mutations in VOCs. Our dynamics analysis also provides a mechanistic insight where the Omicron-defining sites have greater control over the binding sites than the Delta variant and are dynamically additive with the functional advantage of CAPs, thus, the greatest infectivity may not be a coincidence.

Specifically, we find that the interactions between CAP sites and VOC-defining mutations show fingerprints of non-additive dynamics within the Delta variant. In contrast, mutations leading to the Omicron variant are largely additive, driving critical dynamical behavior closer to the patterns observed within the wild-type. These interactions may also drive observed behavior similar between the reference and Omicron strains yet differ in the delta strain, such as the severity of infection as evidenced by hospitalization rates (Houhamdi et al., 2022; Menni et al., 2022). It has also been shown that the Omicron variant has a lower binding affinity with hACE2 than previous variants (Wu et al., 2022), which may contribute to its low pathogenicity.

Long-ranged interactions between different sites within a given protein are critically important for protein function (Peters and Lively, 1999; Bershtein et al., 2006; Collins et al., 2006; Ekeberg et al., 2013; Levy et al., 2017; Harrigan et al., 2018; Otten et al., 2018; Rojas Echenique et al., 2019; Shimagaki and Weigt, 2019; de la Paz et al., 2020; Rizzato et al., 2020; Yang et al., 2020; Bisardi et al., 2022) and for the CoV-2 S protein in particular (Zeng et al., 2020; Castiglione et al., 2021; Dong et al., 2021; Garvin et al., 2021; Nielsen et al., 2022; Ramarao-Milne et al., 2022; Rochman et al., 2022; Rodriguez-Rivas et al., 2022). By showing dynamic differences between the interactions of CAPs, which have likely played a major role in allowing the virus to infect human hosts, the binding site, and the characteristic mutations of dominant Delta and Omicron strains, we see a ‘fine-tuning’ of protein behavior. As variants continue to evolve, Omicron subvariants are of growing concern due in large part to further increased immune evasion (Callaway, 2022; Wang et al., 2022a; Wang et al., 2022b), and we observe that the new mutations observed in antibody binding sites yield more epistatic interaction with the CAPs. In addition to supporting previous dynamic research on the S protein, this analysis provides the insight that CAP sites are of continued importance to protein function and should be given special attention when considering the impact of future mutations.

### Methods

### Dynamic Flexibility and Dynamic Coupling

The DFI utilizes a PRS technique that combines the elastic network model (ENM) and LRT (Gerek and Ozkan, 2011; Nevin Gerek et al., 2013). In ENM, the protein is considered as a network of beads at Ca positions interacting with each other via a harmonic spring potential. Using LRT, ΔR is calculated as the fluctuation response vector of residue j due to unit force's F perturbation on residue i, averaged over multiple unit force directions to simulate an isotropic perturbation.

$${[ Δ R ]}_{\text{?} N \times 1} = {{[ H ]}_{\text{?} N \times \text{?} N}^{- 1} [ F ]}_{\text{?} N \times 1} . \qquad Eq . 1$$

$$\text{?} \text{indicates text missing or illegible when filed}$$

where H is the Hessian, a 3N×3N matrix that can be constructed from 3-D atomic coordinate information and is composed of the second derivatives of the harmonic potential with respect to the components of the position's vectors of length 3N. The Hessian inverse in this equation may be replaced with the covariance matrix G obtained from MD simulations as follows:

$${[ Δ R ]}_{\text{?} N \times 1} = {{[ G ]}_{\text{?} N \times \text{?} N} [ F ]}_{\text{?} N \times 1} . \qquad Eq . 2$$

$$\text{?} \text{indicates text missing or illegible when filed}$$

MD simulations were used to obtain the DFI profiles of Omicron, Omicron XBB, and Omicron XBB 1.5. In order to obtain DFI, each position in the structure was perturbed sequentially to generate a Perturbation Response Matrix A

$${A}_{N \times N} = [ \begin{matrix}{\vert Δ {R}^{1} \vert}_{1} & … & {\vert Δ {R}^{N} \vert}_{1} \\ ⋮ & ⋱ & ⋮ \\ {\vert Δ {R}^{1} \vert}_{N} & … & {\vert Δ {R}^{N} \vert}_{N}\end{matrix} ] . \qquad Eq . 3$$

Where |ΔR^{j}|ᵢ=\sqrt{ [symbol](ΔR)² [symbol]} is the magnitude of fluctuation response at position i due to perturbations at position j. The DFI value of position i is then treated as the displacement response of position i relative to the net displacement response of the entire protein, which is calculated by sequentially perturbing each position in the structure.

$${DFI}_{i} = \frac{{\sum }_{j = 1}^{N} {\vert Δ {R}^{\text{?}} \vert}_{\text{?}}}{{\sum }_{\text{?}}^{N} {\sum }_{j = 1}^{N} {\vert Δ {R}^{\text{?}} \vert}_{\text{?}}} . \qquad Eq . 4$$

$$\text{?} \text{indicates text missing or illegible when filed}$$

It is also often useful to quantify position flexibility relative to the flexibility ranges unique to individual structures. To that end, DFI can be presented as a percentile rank, % DFI. All % DFI calculations present in this work used the DFI value of every residue of the full spike structure for ranking. The DFI parameter can be considered a measure of a given amino acid position's ability to explore its local conformational space.

### Dynamic Coupling Index

Similar to DFI, the DCI (Larrimore et al., 2017; Kumar et al., 2015) also utilizes PRS with the ENM and LRT. DCI captures the strength of displacement response of a given position i upon perturbation to a single functionally important position (or subset of positions) j, relative to the average fluctuation response of position i when all of the positions within a structure are perturbed.

$${DCI}_{\text{?}} = \frac{{\sum }_{j}^{{N}_{\text{?}}} {\vert Δ {R}^{j} \vert}_{\text{?}} / {N}_{\text{?}}}{{\sum }_{j = 1}^{N} {\vert Δ {R}^{\text{?}} \vert}_{\text{?}} / N} . \qquad Eq . 5$$

$$\text{?} \text{indicates text missing or illegible when filed}$$

When only positional pairs are concerned, this expression reduces to

$${DCI}_{\text{?}} = \frac{{\vert Δ {R}^{\text{?}} \vert}_{\text{?}}}{{\sum }_{\text{?}}^{N} {\vert Δ {R}^{\text{?}} \vert}_{\text{?}} / N} . \qquad Eq . 6$$

$$\text{?} \text{indicates text missing or illegible when filed}$$

As such, this parameter represents a measure of the dynamic coupling between i and j upon a perturbation to j. As with DFI, DCIji can also be presented as a percentile-ranked % DCIji.

One of the most important aspects of DCI is that the entire network of interactions is explicitly included in subsequent calculations without the need for dimensionality reduction techniques. If one considers interactions such as communication directionality or dynamic coupling regulation between position pairs as inherent properties of an anisotropic interaction network, it is critical to include the interactions of the entire network to accurately model the effect one residue can have on another.

Here, we present two further extensions of DCI, which allow us to uniquely model coupling directionality and epistatic effects: DCI_{asym} and EpiScore, respectively. Interestingly, we can capture asymmetry between different residues within a protein through DCI, as a coupling in and of itself is asymmetric within an anisotropic network. That is, each amino acid has a set of positions to which it is highly coupled, and this anisotropy in connections gives rise to unique differences in coupling between a given i j pair of amino acids which do not have direct interactions (FIG. 2A). DCI_{asym}, then, is simply DCIᵢⱼ (the normalized displacement response of position j upon a perturbation to position i)—DCIⱼᵢ (Equation (7)). Using DCI_{asym}, we can determine a cause-effect relationship between the i j pair in terms of force/signal propagation between these two positions.

$${DCI}_{asym} = {DCI}_{ij} - {DCI}_{ji} . \qquad Eq . 7$$

$$\% {DCI}_{asym} = \% {DCI}_{ij} - \% {DCI}_{ji} . \qquad Eq . 8$$

where a positive DCI_{asym} value indicates communication from position i to position j.

EpiScore can identify or describe potential non-additivity in substitution behavior between residue pairs. This metric can capture the differences in a normalized perturbation response to a position k when a force is applied at two residues i and j simultaneously versus the average additive perturbation response when each residue i, j, is perturbed individually (FIG. 5A, Eq. 9).

$$EpiScore = \frac{\% {DCI}_{\text{?}}}{\frac{1}{2} ( \% {DCI}_{ik} + \% {DCI}_{jk} )} . \qquad Eq . 9$$

$$\text{?} \text{indicates text missing or illegible when filed}$$

EpiScore values<1 (>1) indicate that the additive perturbations of positions i and j generate a greater (lesser) response at position k than the effect of a simultaneous perturbation. This means that, when treated with a simultaneous perturbation at both sites i and j, the displacement response of k is lower (higher) than the average effect of individual perturbations to i and j, one at a time. As EpiScore is a linear scale, the further the value from 1, the greater the effect described above.

### Molecular Dynamics (MD)

The production simulations for the Omicron variants, including Omicron, Omicron XBB, and Omicron XBB 1.5, were performed with the AMBER software package. These variants, each characterized by specific mutations, were modeled based on the template PDB structure 6M0J. The initial protein configurations in the simulations were parameterized using the ff14SB force field (Maier et al., 2015). In order to create an appropriate solvation environment for the proteins, a solvation box was defined around them, maintaining a minimum separation distance of 16 Å from the protein to the box boundaries. This was accomplished by employing the explicit TIP3P water model (Sun and Kollman, 1995), with the addition of sodium and chloride ions to maintain overall charge neutrality.

The simulation procedure involves an initial energy minimization step, aimed at mitigating steric clashes and optimizing the system's energy. The steepest descent algorithm was applied, encompassing 11,000 steps. Subsequently, the system underwent a gradual temperature increase (heat up), up to 300 K, and was subjected to production simulations under a constant number of particles, pressure, and temperature ensemble (NPT).

During these production simulations, temperature was maintained at 300 K, with pressure regulation set at 1 bar. Temperature regulation was achieved through the utilization of the Langevin thermostat (Hünenberger, 2005) and Berendsen barostat (Berendsen et al., 1984), featuring a collision frequency of 1.0 picoseconds⁻¹. Hydrogen atom bond lengths were constrained using the SHAKE algorithm (Pearlman et al., 1995). The production trajectories were simulated for 1 μs each.

To ensure the reliability of the simulations and assess their convergence, a convergence criterion was employed. The achieved convergence was determined by monitoring the root mean square deviation (RMSD) between the highest sampled conformation in consecutive time windows (Sawle and Ghosh, 2016). Specifically, convergence was defined as the point at which the RMSD between the highest sampled conformation in the last 300 ns window and the 300 ns window immediately preceding it dropped below 1 Å. Window sizes varying from 100 ns to 500 ns were employed to evaluate convergence, ensuring the robustness and stability of the obtained results.

To calculate DFI, covariance matrix data were computed over different time windows as discussed above. By default, utilizing the Hessian implies a restriction to a harmonic potential, assuming that the data are sampled from a Gaussian distribution. Ergodicity in both simulation time and initial structures sampled in each time interval ensures two key conditions: (i) consistency of potential energy across conformations sampled from the same distribution. (ii) The sampling of different initial conformations while computing covariance matrices at various time windows eliminates global motions and accurately captures equilibrium coordinates. Consequently, the final average DFI profiles are independent of time window size, resulting in consistent results across different time window sizes (e.g., 50 ns vs. 75 ns) and enabling the acquisition of statistically significant DFI values.

### Statistical Tests

Pearson correlation coefficients (r) were used to demonstrate linear relationships between continuous variables in FIG. 3 and FIGS. 8A-8E. Student's independent t-tests were performed to demonstrate a significant difference between distributions in FIGS. 2A-2B, FIGS. 4A-4B and FIGS. 6A-6C (as well as Table 1 and Table 2), FIGS. 6A-6C), and FIGS. 7A-7B as demonstrated by the p-value.

### REFERENCES FOR EXAMPLE 1

Abduljalil J M, Elghareib A M, Samir A, Ezat A A, Elfiky A A. (2023) How helpful were molecular dynamics simulations in shaping our understanding of SARS-CoV-2 spike protein dynamics? International Journal of Biological Macromolecules 242:125153.
Ali F, Kasry A, Amin M. (2021) The new SARS-CoV-2 strain shows a stronger binding affinity to ACE2 due to N501Y mutant. Medicine in Drug Discovery 10:100086.
Amicone M, et al. (2022) Mutation rate of SARS-CoV-2 and emergence of mutators during experimental evolution. Evolution, Medicine, and Public Health 10:142-155.
Amicone M, et al. (2022) Mutation rate of SARS-CoV-2 and emergence of mutators during experimental evolution. Evolution, Medicine, and Public Health 10:142-155.
Bager P, et al. (2021) Hospitalisation associated with SARS-CoV-2 delta variant in Denmark. The Lancet. Infectious Diseases 21:1351.
Barton M I, et al. (2021) Effects of common mutations in the SARS-CoV-2 Spike RBD and its ligand, the human ACE2 receptor on binding affinity and kinetics. eLife 10:e70658.
Berendsen H J C, et al. (1984) Molecular dynamics with coupling to an external bath. The Journal of Chemical Physics 81:3684-3690.
Berman H M, et al. (2000) The protein data bank. Nucleic Acids Research 28:235-242.
Bershtein S, et al. (2006). Robustness-epistasis link shapes the fitness landscape of a randomly drifting protein. Nature 444:929-932.
Bhabha G, et al. (2013) Divergent evolution of protein conformational dynamics in dihydrofolate reductase. Nature Structural and Molecular Biology 20:1243-1249.
Bisardi M, et al. (2022) Modeling sequence-space exploration and emergence of epistatic signals in protein evolution. Molecular Biology and Evolution 39:msab321.
Boni M F, et al. (2020) Evoluationary origins of the SARS-CoV-2 sarbecovirus lineage responsible for the COVID-10 pandemic. Nature Microbiology 5:1408-1417.
Brister J R, et al. (2015) NCBI viral genomes resource. Nucleic Acids Research 43:D571-D577.
Butler B M et al. (2018) Coevolving residues inform protein dynamics profiles and disease susceptibility of nSNVs. PLOS Computational Biology 14:e1006626.
Cagliani R, et al. (2020) Computational inference of selection underlying the evolution of the novel Coronavirus, severe acute respiratory syndrome Coronavirus 2. Journal of Virology 94:e00311-20.
Callaway E (2022) COVID ‘variant soup’ is making winter surges hard to predict. Nature 611:213-214.
Campbell E, et al. (2016) The role of protein dynamics in the evolution of new enzyme function. Nature Chemical Biology 12:944-950.
Campitelli P, et al. (2020) The role of conformational dynamics and allostery in modulating protein evolution. Annual Review of Biophysics 49:267-288.
Campitelli P, Ozkan S B. (2020) Allostery and epistasis: emergent properties of anisotropic networks. Entropy 22:667.
Campitelli P, et al. (2021) Substitutions at nonconserved rheostat positions modulate function by rewiring long-range, dynamic interactions. Molecular Biology and Evolution 38:201-214.
Campitelli P, et al. (2022) Dynamic allostery highlights the evolutionary differences between CoV-1 and CoV-2 main proteases. Biophysical Journal 121:1483-1492.
Cantoni D, et al. (2022) Evolutionary remodeling of N-terminal domain loops fine-tunes SARS-CoV-2 spike. EMBO Reports 23:e54322.
Carabelli A M, et al. (2023) SARS-SoV-2 variant biology: immune escape, transmission and fitness. Nature Reviews Microbiology 21:162-177.
Castiglione G M, et al. (2021) Evolutionary pathways to SARS-CoV-2 resistance are opened and closed by epistasis acting on ACE2. PLOS Biology 19:e3001510.
Chan C M, et al. (2008) RETRACTED: spike protein, s, of human coronavirus HKU1: role in viral life cycle and application in antibody detection. Experimental Biology and Medicine 233:1527-1536.
Changeux J P, Edelstein S J (2005) Allosteric mechanisms of signal transduction. Science 308:1424-1428.
Chi X, et al. (2020) A neutralizing human antibody binds to the N-terminal domain of the Spike protein of SARS-CoV-2. Science 369:650-655.
Collins S R, et al. (2006) A strategy for extracting and analyzing large-scale quantitative epistatic interaction data. Genome Biology 7:R63.
Damas J et al. (2020) Broad host range of SARS-CoV-2 predicted by comparative and structural analysis of ACE2 in vertebrates PNAS 117:22311-22322.
de la Paz J A, et al. (2020) Epistatic contributions promote the unification of incompatible models of neutral molecular evolution. PNAS 117:5873-4882.
Deng X, et al. (2021) Transmission, infectivity, and antibody neutralization of an emerging SARS-CoV-2 variant in California carrying a L452R Spike protein mutation. medRxiv.
Diaz-Salinas M A, et al. (2022) Conformational dynamics and allosteric modulation of the SARS-CoV-2 spike. eLife 11:E75433.
Dicken S J, et al. (2021) Characterisation of B.1.1.7 and pangolin coronavirus spike provides insights on the evolutionary trajectory of SARS-CoV-2. bioRxiv.
Dong A, et al. (2021) The genomic physics of COVID-19 pathogenesis and spread. Cells 11:80.
Doshi U, et al. (2016) Dynamical network of residue-residue contacts reveals coupled allosteric effects in recognition, catalysis, and mutation. PNAS 113:4735-4740.
Dror R O, et al. (2012) Biomolecular simulation: A computational microscope for molecular biology. Annual Review of Biophysics 41:429-452.
Earnest, R, et al. (2022) Comparative transmissibility of SARS-CoV-2 variants Delta and Alpha in New England, USA Cell Reports. Medicine 3:100583.
Ekeberg M, et al. (2013) Improved contact prediction in proteins: Using pseudolikelihoods to infer Potts models Physical Review E 87:012707.
Fraser B J, et al. (2022) Structure and activity of human TMPRSS2 protease implicated in SARS-CoV-2 activation Nature Chemical Biology 18:963-971.
Frost S D W, et al. (2018) Neutral theory and rapidly evolving viral pathogens Molecular Biology and Evolution 35:1348-1354.
Garvin M R, et al. (2021) Rapid Expansion of SARS-CoV-2 variants of concern is a result of adaptive epistasis bioRxiv.
Gerek Z N, Ozkan S B. Change in allosteric network affects binding affinities of PDZ domains PLOS Computational Biology 7:e1002154.
Gobeil S M C, et al. (2021a) D614G mutation alters SARS-CoV-2 spike conformation and enhances protease cleavage at the S1/S2 junction Cell Reports 34:108630.
Gobeil S M G, et al. (2021b) Effect of natural mutations of SARS-CoV-2 on spike structure, conformation, and antigenicity Science 373:eabi6226.
Gur M, et al. (2020) Conformational transition of SARS-CoV-2 spike glycoprotein between its closed and open states The Journal of Chemical Physics 153:075101.
Harrigan P, et al. (2018) Real-time genetic compensation defines the dynamic demands of feedback control Cell 175:877-886.
Harvey W T, et al. (2021) SARS-CoV-2 variants, spike mutations and immune escape Nature Reviews. Microbiology 19:409-424.
Henderson R, et al. (2020) Controlling the SARS-CoV-2 spike glycoprotein conformation Nature Structural & Molecular Biology 27:925-933.
Hoffmann M, et al. (2020) SARS-CoV-2 cell entry depends on ACE2 and TMPRSS2 and Is blocked by a clinically proven protease inhibitor Cell 181:271-280.
Hong Q, et al. (2022) Molecular basis of receptor binding and antibody neutralization of Omicron Nature 604:546-552.
Houhamdi L, et al. (2022) Characteristics of the first 1119 SARS-CoV-2 Omicron variant cases Journal of Medical Virology 94:2290-2295.
Huang, Y, et al. (2020) Structural and functional properties of SARS-CoV-2 spike protein: potential antivirus drug development for COVID-19 Acta Pharmacologica Sinica 41:1141-1149.
Hunenberger P H. (2005) Thermostat Algorithms for molecular Dynamics simulations In: Holm C, Kremer K, editors. Advanced Computer Simulation. Springer. pp. 105-149.
Jackson C B, et al. (2022) Mechanisms of SARS-CoV-2 entry into cells Nature Reviews. Molecular Cell Biology 23:3-20.
Kazan I C, et al. (2022) Design of novel cyanovirin-N variants by modulation of binding dynamics through distal mutations eLife 11:eLife.
Kazan I C, et al. (2023) Allosteric regulatory control in dihydrofolate reductase is revealed by dynamic asymmetry Protein Science 32:e4700.
Kemp S A et al. (2021) SARS-CoV-2 evolution during treatment of chronic infection Nature 592:277-282.
Keskin O, et al. (2000) Characterization of anticancer agents by their growth inhibitory activity and relationships to mechanism of action and structure Anti-Cancer Drug Design 15:79-98.
Khan A, et al. (2021) Higher infectivity of the SARS-CoV-2 new variants is associated with K417N/T, E484K, and N501Y mutants: An insight from structural data Journal of Cellular Physiology 236:7045-7057.
Kim H, et al. (2015) A hinge migration mechanism unlocks the evolution of green-to-red photoconversion in GFP-like proteins Structure 23:34-43.
Kim S, et al. (2021) SARS-CoV-2 omicron mutation is faster than the chase: multiple mutations on Spike/ACE2 interaction residues Immune Network 21:e38.
Kimura M (1983) The neutral theory of molecular evolution Cambridge University Press.
Kirchdoerfer R N, et al. (2016) Pre-fusion structure of a human coronavirus spike protein Nature 531:118-121.
Kistler K E, et al. Rapid and parallel adaptive mutations in spike S1 drive clade success in SARS-CoV-2 Cell Host & Microbe 30:545-555.
Klinakis A, et al. (2021) N-terminal domain mutations of the spike protein are structurally implicated in epitope recognition in emerging SARS-CoV-2 strains Computational and Structural Biotechnology Journal 19:5556-5567.
Kolbaba-Kartchner B, et al. (2021) The role of rigid residues in modulating TEM-1 (3-lactamase function and thermostability International Journal of Molecular Sciences 22:2895.
Kumar A, et al. (2015) The role of conformational dynamics and allostery in the disease development of human ferritin Biophysical Journal 109:1273-1281.
Kumar S, Patel R. (2018) Neutral theory, disease mutations, and personal exomes Molecular Biology and Evolution 35:1297-1303.
Kumar S, et al. (2018) MEGA X: molecular evolutionary genetics analysis across computing platforms Molecular Biology and Evolution 35:1547-1549.
Kumar S, et al. (2021) An evolutionary portrait of the progenitor SARS-CoV-2 and Its dominant offshoots in COVID-19 pandemic Molecular Biology and Evolution 38:3046-3059.
Kuzmanic A, et al. (2020) Investigating cryptic binding sites by molecular dynamics simulations Accounts of Chemical Research 53:654-661.
Labbadia J, Morimoto R I. (2015) The biology of proteostasis in aging and disease Annual Review of Biochemistry 84:435-464.
Laiton-Donato K, et al. (2021) Characterization of the emerging B.1.621 variant of interest of SARS-CoV-2 Infection, Genetics and Evolution 95:105038.
Lan J, et al. (2020) Structure of the SARS-CoV-2 spike receptor-binding domain bound to the ACE2 receptor Nature 581:215-220.
Larrimore K E, et al. (2017) Plant-expressed cocaine hydrolase variants of butyrylcholinesterase exhibit altered allosteric effects of cholinesterase activity and increased inhibitor sensitivity Scientific Reports 7:10419.
Levy R M, et al. (2017) Potts Hamiltonian models of protein co-variation, free energy landscapes, and evolutionary fitness Current Opinion in Structural Biology 43:55-62.
Liu Y, Bahar I. (2012) Sequence evolution correlates with structural dynamics Molecular Biology and Evolution 29:2253-2263.
Liu L, et al. (2016) A molecular evolutionary reference for the human variome Molecular Biology and Evolution 33:245-254.
Liu X, et al. (2022) Rampant C-to-U deamination accounts for the intrinsically high mutation rate in SARS-CoV-2 spike gene RNA 28:917-926.
Ma B, Nussinov R. (2016) Conformational footprints Nature Chemical Biology 12:890-891.
Maguid S, et al. (2006) Evolutionary conservation of protein backbone flexibility Journal of Molecular Evolution 63:448-457.
Maguid S, et al. (2008) Evolutionary conservation of protein vibrational dynamics Gene 422:7-13.
Maher M C, et al. (2022) Predicting the mutational drivers of future SARS-CoV-2 variants of concern Science Translational Medicine 14:eabk3445.
Maier J A, et al. (2015) ff14S B: improving the accuracy of protein side chain and backbone parameters from ff99S B Journal of Chemical Theory and Computation 11:3696-3713.
Majumder S, et al. (2021) Exploring the intrinsic dynamics of SARS-CoV-2, SARS-CoV and MERS-CoV spike glycoprotein through normal mode analysis using anisotropic network model Journal of Molecular Graphics & Modelling 102:107778.
Markov P V, et al. (2023) The evolution of SARS-CoV-2 Nature Reviews Microbiology 21:361-379.
Menni C, et al. (2022) Symptom prevalence, duration, and risk of hospital admission in individuals infected with SARS-CoV-2 during periods of omicron and delta variant dominance: a prospective observational study from the ZOE COVID Study The Lancet 399:1618-1624.
Mikulska-Ruminska K, et al. (2019) Characterization of differential dynamics, specificity, and allostery of lipoxygenase family members Journal of Chemical Information and Modeling 59:2496-2508.
Millet J K, Whittaker G R. (2014) Host cell entry of Middle East respiratory syndrome coronavirus after two-step, furin-mediated activation of the spike protein PNAS 111:15214-15219.
Millet J K, Whittaker G R. (2015) Host cell proteases: Critical determinants of coronavirus tropism and pathogenesis Virus Research 202:120-134.
Mishra S K, Jernigan R L. (2018) Protein dynamic communities from elastic network models align closely to the communities defined by molecular dynamics PLOS ONE 13:e0199225.
Modi T, Ozkan S B. (2018) Mutations utilize dynamic allostery to confer resistance in TEM-1 β-lactamase International Journal of Molecular Sciences 19:3808.
Modi T, et al. (2021a) Protein folding stability and binding interactions through the lens of evolution: a dynamical perspective Current Opinion in Structural Biology 66:207-215.
Modi T, et al. (2021b) Hinge-shift mechanism as a protein design principle for the evolution of β-lactamases from substrate promiscuity to specificity Nature Communications 12:1852.
Moulana A, et al. (2022) Compensatory epistasis maintains ACE2 affinity in SARS-CoV-2 Omicron BA.1 Nature Communications 13:7011.
Moulana A, et al. (2023) The landscape of antibody binding affinity in SARS-CoV-2 Omicron BA.1 evolution eLife 12:e83442.
Neher R A (2022) Contributions of adaptation and purifying selection to SARS-CoV-2 evolution Virus Evolution 8:veac113.
Nevin Gerek Z, et al. (2013) Structural dynamics flexibility informs function and evolution at a proteome scale Evolutionary Applications 6:423-433.
Nguyen H L, et al. (2020) Does SARS-CoV-2 bind to human ACE2 More strongly than does SARS-CoV? The Journal of Physical Chemistry. B 124:7336-7347.
Nielsen B F, et al. (2022) Immune heterogeneity and epistasis explain punctuated evolution of SARS-CoV-2 bioRxiv.
Nussinov R, Tsai C J (2013) Allostery in disease and in drug discovery Cell 153:293-305.
O'Rourke K F, et al. (2016) Biophysical and computational methods to analyze amino acid interaction networks in proteins Computational and Structural Biotechnology Journal 14:245-251.
Ose N J, et al. (2022a) Dynamic coupling of residues within proteins as a mechanistic foundation of many enigmatic pathogenic missense variants PLOS Computational Biology 18:e1010006.
Ose N J, et al. (2022b) Protein dynamics provide mechanistic insights about the epistatic relationships among highly observed potentially adaptive missense variants Biophysical Journal 121:456a.
Otten R, et al. (2018) Rescue of conformational dynamics in enzyme catalysis by directed evolution Nature Communications 9:1314.
Ozkan S B (2024) Dfi-DCI, version swh:1:rev:7a3a54bbe03e8356036bdb164ad1b2c01c103cc5 Software Heritage.
Ozono S, et al. (2021) SARS-CoV-2 D614G spike mutation increases entry efficiency with enhanced ACE2-binding affinity Nature Communications 12:848.
Pearlman D A, et al. (1995) AMBER, a package of computer programs for applying molecular mechanics, normal mode analysis, molecular dynamics and free energy calculations to simulate the structural and energetic properties of molecules Computer Physics Communications 91:1-41.
Peters A D, Lively C M. (1999) The red queen and fluctuating epistasis: a population genetic analysis of antagonistic coevolution The American Naturalist 154:393-405.
Pipito L, et al. (2022) Molecular dynamics studies reveal structural and functional features of the SARS-CoV-2 spike protein BioEssays 44:e2200060.
Qu P, et al. (2023) Extraordinary evasion of neutralizing antibody response by omicron XBB.1.5, CH.1.1 and CA.3.1 variants bioRxiv.
Raghuvamsi P V, et al. (2021) SARS-CoV-2 S protein:ACE2 interaction reveals novel allosteric targets eLife 10:e63646.
Ramarao-Milne P, et al. (2022) Data-driven platform for identifying variants of interest in COVID-19 virus Computational and Structural Biotechnology Journal 20:2942-2950.
Rehman S U, et al. (2020) Evolutionary trajectory for the emergence of novel coronavirus SARS-CoV-2 Pathogens 9:240.
Rizzato F, et al. (2020) Inference of compressed Potts graphical models Physical Review. E 101:012309.
Rochman N D, et al. (2021) Ongoing global and regional adaptive evolution of SARS-CoV-2 PNAS 118:e2104241118.
Rochman N D, et al. (2022) Epistasis at the SARS-CoV-2 receptor-binding domain interface and the propitiously boring implications for vaccine escape mBio 13:e0013522.
Rodriguez-Rivas J, et al. (2022) Epistatic models predict mutable sites in SARS-CoV-2 proteins and epitopes PNAS 119:e2113118119.
Rojas Echenique J I, et al. (2019) Modular epistasis and the compensatory evolution of gene deletion mutants PLOS Genetics 15:e1007958.
Saavedra H G, et al. (2018) Dynamic allostery can drive cold adaptation in enzymes Nature 558:324-328.
Saputri D S, et al. (2020) Flexible, functional, and familiar: characteristics of SARS-CoV-2 Spike protein evolution Frontiers in Microbiology 11:2112.
Sawle L, Ghosh K. (2016) Convergence of molecular dynamics simulation of protein native states: feasibility vs self-consistency dilemma Journal of Chemical Theory and Computation 12:861-869.
Sekhar A, Kay L E. (2019) An NMR view of protein dynamics in health and disease Annual Review of Biophysics 48:297-319.
Shang J, et al. (2020) Cell entry mechanisms of SARS-CoV-2 PNAS 117:11727-11734.
Sheikh A, et al. (2021) SARS-CoV-2 Delta VOC in Scotland: demographics, risk of hospital admission, and vaccine effectiveness The Lancet 397:2461-2462.
Shimagaki K, Weigt M. (2019) Selection of sequence motifs and generative Hopfield-Potts models for protein families Physical Review E 100:032128.
Shoemark D K, et al. (2022) Molecular dynamics of spike variants in the locked conformation: RBD interfaces, fatty acid binding and furin cleavage sites bioRxiv.
Singh D, Yi S V. (2021) On the origin and evolution of SARS-CoV-2 Experimental & Molecular Medicine 53:537-547.
Socher E, et al. (2021) Mutations in the B.1.1.7 SARS-CoV-2 spike protein reduce receptor-binding affinity and induce a flexible link to the fusion peptide Biomedicines 9:525.
Spinello A, et al. (2021) Allosteric cross-talk among spike's receptor-binding domain mutations of the SARS-CoV-2 South African variant triggers an effective hijacking of human cell receptor The Journal of Physical Chemistry Letters 12:5987-5993.
Starr T N, et al. (2022a) Shifting mutational constraints in the SARS-CoV-2 receptor-binding domain during viral evolution Science 377:420-424.
Starr T N, et al. (2022b) Deep mutational scans for ACE2 binding, RBD expression, and antibody escape in the SARS-CoV-2 Omicron BA.1 and BA.2 receptor-binding domains PLOS Pathogens 18:e1010951.
Starr T N, et al. (2022c) ACE2 binding is an ancestral and evolvable trait of sarbecoviruses Nature 603:913-918.
Steinhauer D A (1999) Role of hemagglutinin cleavage for the pathogenicity of influenza virus Virology 258:1-20.
Stevens A O, et al. (2022) Investigating the allosteric response of the PICK1 PDZ domain to different ligands with all-atom simulations Protein Science 31:e4474.
Sun Y, Kollman P A (1995) Hydrophobic solvation of methane and nonbond parameters of the TIP3P water model Journal of Computational Chemistry 16:1164-1169.
Swint-Kruse L, et al. (1998) Comparison of simulated and experimentally determined dynamics for a variant of the Lacl DNA-binding domain, Nlac-P Biophysical Journal 74:413-421.
Sztain T, et al. (2021) A glycan gate controls opening of the SARS-CoV-2 spike protein Nature Chemistry 13:963-968.
Tan Z W, et al. (2022) Allosteric perspective on the mutability and druggability of the SARS-CoV-2 Spike protein Structure 30:590-607.
Tang X, et al. (2020) On the origin and continuing evolution of SARS-CoV-2 National Science Review 7:1012-1023.
Tay J H, et al. (2022) The emergence of SARS-CoV-2 variants of concern is driven by acceleration of the substitution rate Molecular Biology and Evolution 39:msac013.
Teruel N, et al. (2021a) Computational analysis of the effect of SARS-CoV-2 variant omicron spike protein mutations on dynamics, ACE2 binding and propensity for immune escape bioRxiv.
Teruel N, et al. (2021b) Modelling conformational state dynamics and its role on infection for SARS-CoV-2 Spike protein variants PLOS Computational Biology 17:e1009286.
Thye A Y K, et al. (2021) Emerging SARS-CoV-2 Variants of Concern (VOCs): an impending global crisis Biomedicines 9:1303.
Twohig K A, et al. (2022) Hospital admission and emergency care attendance risk for SARS-CoV-2 delta (B.1.617.2) compared with alpha (B.1.1.7) variants of concern: a cohort study The Lancet Infectious Diseases 22:35-42.
Verkhivker G (2022) Allosteric determinants of the SARS-CoV-2 Spike protein binding with nanobodies: examining mechanisms of mutational escape and sensitivity of the omicron variant International Journal of Molecular Sciences 23:2172.
Walls A C, et al. (2020) Structure, function, and antigenicity of the SARS-CoV-2 spike glycoprotein Cell 181:281-292.
Wang Q, et al. (2022a) Antibody evasion by SARS-CoV-2 Omicron subvariants BA.2.12.1, BA.4 and BA.5 Nature 608:603-608.
Wang Q, et al. (2022b) Alarming antibody evasion properties of rising SARS-CoV-2 BQ and XBB subvariants bioRxiv.
Weisblum Y et al. (2020) Escape from neutralizing antibodies by SARS-CoV-2 spike protein variants eLife 9:e61312.
Witte L, et al. (2023) Epistasis lowers the genetic barrier to SARS-CoV-2 neutralizing antibody escape Nature Communications 14:302.
Wrapp D, et al. (2020) Cryo-EM structure of the 2019-nCoV spike in the prefusion conformation Science 367:1260-1263.
Wrobel A G, et al. (2020) SARS-CoV-2 and bat RaTG13 spike glycoprotein structures inform on virus evolution and furin-cleavage effects Nature Structural & Molecular Biology 27:763-767.
Wu L, et al. (2022) SARS-CoV-2 Omicron RBD shows weaker binding affinity than the currently dominant Delta variant to human ACE2 Signal Transduction and Targeted Therapy 7:8.
Xue Q, et al. (2022) Computational insights into the allosteric effect and dynamic structural features of the SARS-COV-2 spike protein Chemistry 28:e202104215.
Yang Q E, et al. (2020) Compensatory mutations modulate the competitiveness and dynamics of plasmid-mediated colistin resistance in Escherichia coli clones The ISME Journal 14:861-865.
Yue C, et al. (2023) Enhanced transmissibility of XBB.1.5 is contributed by both strong ACE2 binding and antibody evasion bioRxiv.
Yurkovetskiy L, et al. (2020) Structural and functional analysis of the D614G SARS-CoV-2 spike protein variant Cell 183:739-751.
Zeng H L, et al. (2020) Global analysis of more than 50,000 SARS-CoV-2 genomes reveals epistasis between eight viral genes PNAS 117:31519-31526.
Zhang J, et al. (2021) Structural impact on SARS-CoV-2 spike protein by D614G substitution Science 372:525-530.
Zhou T, et al. (2020) Cryo-EM structures of SARS-CoV-2 spike without and with ACE2 reveal a pH-dependent switch to mediate endosomal positioning of receptor-binding domains Cell Host & Microbe 28:867-879.

### Tables for Example 1

TABLE 1

| | 18 | 19 | 20 | 26 | 27 | 67 | 80 | 83 | 95 | 138 | 142 | 145 | 146 | 158 | 183 | 190 | 212 |
|---|---|---|---|---|---|---|---|---|---|---|---|---|---|---|---|---|---|
| Wild-type | L | T | T | P | A | A | D | V | T | D | G | Y | H | R | Q | R | L |
| (MN90894.3) | | | | | | | | | | | | | | | | | |
| Alpha | L | T | T | P | A | A | D | V | T | D | G | Y | H | R | Q | R | L |
| Beta | L | T | T | P | A | A | A | V | T | D | G | Y | H | R | Q | R | L |
| Gamma | F | T | N | S | A | A | D | V | T | Y | G | Y | H | R | Q | S | L |
| Delta | L | R | T | P | A | A | D | V | T | D | G | Y | H | G | Q | R | L |
| Omicron | L | T | T | P | A | V | D | V | I | D | G | D | H | R | Q | R | I |
| Omicron | L | I | T | P | S | A | D | A | T | D | D | Y | Q | R | E | R | L |
| XBB 1.5 | | | | | | | | | | | | | | | | | |

| | 213 | 215 | 252 | 339 | 346 | 368 | 371 | 373 | 375 | 376 | 405 | 408 | 417 | 440 | 445 |
|---|---|---|---|---|---|---|---|---|---|---|---|---|---|---|---|
| Wild-type | V | D | G | G | R | L | S | S | S | T | D | R | K | N | V |
| (MN90894.3) | | | | | | | | | | | | | | | |
| Alpha | V | D | G | G | R | L | S | S | S | T | D | R | K | N | V |
| Beta | V | G | G | G | R | L | S | S | S | T | D | R | N | N | V |
| Gamma | V | D | G | G | R | L | S | S | S | T | D | R | T | N | V |
| Delta | V | D | G | G | R | L | S | S | S | T | D | R | K | N | V |
| Omicron | V | D | G | D | R | L | L | P | F | T | D | R | N | K | V |
| Omicron XBB | E | D | V | H | T | I | F | P | F | A | N | S | N | K | P |
| 1.5 | | | | | | | | | | | | | | | |

TABLE 2

| | 446 | 452 | 460 | 477 | 478 | 484 | 486 | 490 | 493 | 496 | 498 | 501 | 505 | 547 | 570 | 614 |
|---|---|---|---|---|---|---|---|---|---|---|---|---|---|---|---|---|
| Wild-type | G | L | N | S | T | E | F | F | Q | G | Q | N | Y | T | A | D |
| (MN90894.3) | | | | | | | | | | | | | | | | |
| Alpha | G | L | N | S | T | E | F | F | Q | G | Q | Y | Y | T | D | G |
| Beta | G | L | N | S | T | K | F | F | Q | G | Q | Y | Y | T | A | G |
| Gamma | G | L | N | S | T | K | F | F | Q | G | Q | Y | Y | T | A | G |
| Delta | G | R | N | S | K | E | F | F | Q | G | Q | N | Y | T | A | G |
| Omicron | S | L | N | N | K | A | F | F | R | S | R | Y | H | K | A | G |
| Omicron XBB | S | L | K | N | K | A | P | S | Q | G | R | Y | H | T | A | G |
| 1.5 | | | | | | | | | | | | | | | | |
| | 655 | 679 | 681 | 701 | 716 | 764 | 796 | 856 | 950 | 954 | 969 | 981 | 982 | 1027 | 1118 | 1176 |
| Wild-type | H | N | P | A | T | N | D | N | D | Q | N | L | S | T | D | V |
| (MN90894.3) | | | | | | | | | | | | | | | | |
| Alpha | H | N | H | A | I | N | D | N | D | Q | N | L | A | T | H | V |
| Beta | H | N | P | V | T | N | D | N | D | Q | N | L | S | T | D | V |
| Gamma | Y | N | P | A | T | N | D | N | D | Q | N | L | S | I | D | F |
| Delta | H | N | R | A | T | N | D | N | N | Q | N | L | S | T | D | V |
| Omicron | Y | K | H | A | T | K | Y | K | D | H | K | F | S | T | D | V |
| Omicron XBB | Y | K | H | A | T | K | Y | N | D | H | K | L | S | T | D | V |
| 1.5 | | | | | | | | | | | | | | | | |

Table 1 and Table 2: A comparison of the amino acid sequences for different variant spike proteins used in this article. Sequence MN90894.3 is used as the wild-type (reference) sequence. For each different variant, residue positions matching the reference sequence are shown in regular font, while locations where the residue differs from the reference sequence are shown in bold. For S protein locations not shown here, all variants presented have the same amino acid type as the reference sequence.

### Example 2: Illustrative Embodiments of Presented Methods

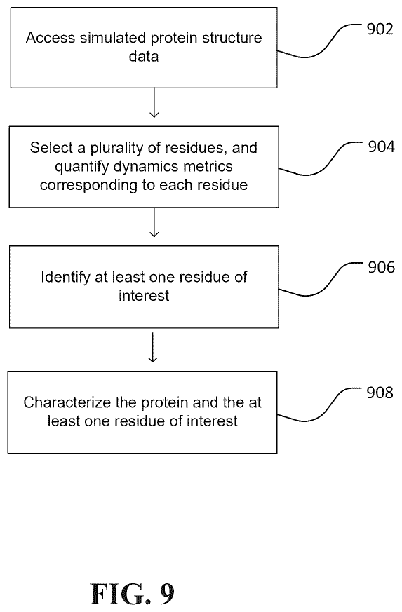

FIG. 9 shows an example process 900 for characterizing a protein. The process may be enacted by using a computer system to execute the steps. At step 902, the process may include accessing simulated protein structure data. At step 904, the process may include selecting a plurality of residues, and quantifying a plurality of dynamics metrics for each residue in the plurality of residues. At step 906, the process may include identifying at least one residue of interest among the plurality of residues. At step 908, the process may then include characterizing the protein and the at least one residue of interest. The process may further include generating a report.

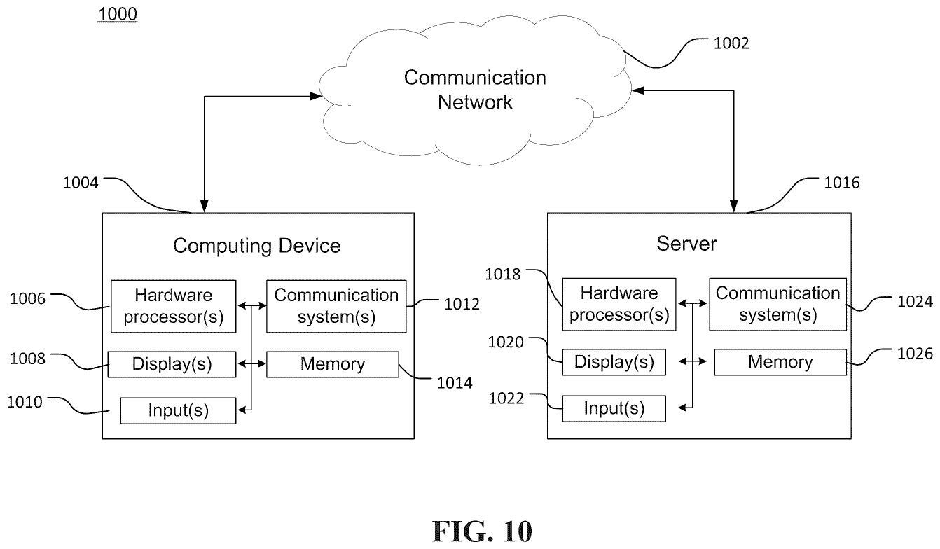

In FIG. 10, an example 1000 of a system (e.g., a data processing system) in accordance with some embodiments of the disclosed subject matter is shown.

In some embodiments, computing device 1004 and/or server 1016 can be any suitable computing device or combination of devices, such as a desktop computer, a laptop computer, a smartphone, a tablet computer, a wearable computer, a server computer, a virtual machine being executed by a physical computing device, etc. As described herein, system 1000 can present information about the protein, dynamics metrics, and/or residue of interest to a user (e.g., a researcher and/or a physician).

In some embodiments, communication network 1002 can be any suitable communication network or combination of communication networks. In some embodiments, communication network 1002 can be any suitable communication network or combination of communication networks. For example, communication network 1002 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 1602 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, etc.

FIG. 10 additionally shows an example of hardware that can be used to implement computing device 1004 and server 1016 in accordance with some embodiments of the disclosed subject matter. In some embodiments, computing device 1004 can be used to execute one or more set of instructions to characterize a protein. In other embodiments, computing device 1004 can be used to calculate an evolutionary probability score for each residue in the plurality of residues. In still other embodiments, computing device 1004 can be used to compare characterization of a protein and a protein variant (e.g., the same protein present in a viral variant).

As shown in FIG. 10, computing device 1004 can include one or more hardware processor 1006, one or more displays 1008, one or more inputs 1010, one or more communications 1012, and/or memory 1014. In some embodiments, processor 1006 can be any suitable hardware processor or combination of processors, such as central processing unit, a graphics processing unit, etc. In some embodiments, display 1008 can include any suitable display devices, such as a computer monitor, a touchscreen, a television, etc. In some embodiments, inputs 1010 can include any suitable input device and/or sensors that can be used to receive user input, such as a keyboard, a mouse, a touchscreen, a microphone, etc.

In some embodiments, communication systems 1012 can include any suitable hardware, firmware, and/or software for communicating information over communication network 1002 and/or any other suitable communication networks. For example, communications systems 1012 can include one or more transceivers, one or more communication chips and/or chip sets, etc. In a more particular example, communications systems 1012 can include hardware, firmware and/or software that can be used to establish a Wi-Fi connection, a Bluetooth connection, a cellular connection, an Ethernet connection, etc.

In some embodiments, memory 1014 can include any suitable storage device or devices that can be used to store instructions, values, etc., that can be used, for example, by processor 1006 to present content using display 1008, to communicate with server 1016 via communications system(s) 1012, etc.

Memory 1014 can include any suitable volatile memory, non-volatile memory, storage, or any suitable combination thereof. For example, memory 1014 can include RAM, ROM, EEPROM, one or more flash drives, one or more hard disks, one or more solid state drives, one or more optical drives, etc. In some embodiments, memory 1014 can have encoded thereon a computer program for controlling operation of computing device 1004. In such embodiments, processor 1006 can execute at least a portion of the computer program to present content (e.g., images, user interfaces, graphics, tables, etc.), receive content from server 1016, transmit information to server 1016, etc.

In some embodiments, server 1016 can include a processor 1018, a display 1020, one or more inputs 1022, one or more communications systems 1024, and/or memory 1026. In some embodiments, processor 1018 can be any suitable hardware processor or combination of processors, such as a central processing unit, a graphics processing unit, etc. In some embodiments, display 1020 can include any suitable display devices, such as a computer monitor, a touchscreen, a television, etc. In some embodiments, inputs 1022 can include any suitable input devices and/or sensors that can be used to receive user input, such as a keyboard, a mouse, a touchscreen, a microphone, etc.

In some embodiments, communications systems 1024 can include any suitable hardware, firmware, and/or software for communicating information over communication network 1002 and/or any other suitable communication networks. For example, communications systems 1024 can include one or more transceivers, one or more communication chips and/or chip sets, etc. In a more particular example, communications systems 1024 can include hardware, firmware and/or software that can be used to establish a Wi-Fi connection, a Bluetooth connection, a cellular connection, an Ethernet connection, etc.

In some embodiments, memory 1026 can include any suitable storage device or devices that can be used to store instructions, values, etc., that can be used, for example, by processor 1018 to present content using display 1020, to communicate with one or more computing devices 1004, etc. Memory 1026 can include any suitable volatile memory, non-volatile memory, storage, or any suitable combination thereof. For example, memory 1026 can include RAM, ROM, EEPROM, one or more flash drives, one or more hard disks, one or more solid state drives, one or more optical drives, etc. In some embodiments, memory 1026 can have encoded thereon a server program for controlling operation of server 1016. In such embodiments, processor 1018 can execute at least a portion of the server program to transmit information and/or content (e.g., results of a tissue identification and/or classification, a user interface, etc.) to one or more computing devices 1004, receive information and/or content from one or more computing devices 1004, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

A number of references to patent and non-patent documents are made throughout the publication, each of which is herein incorporated by reference in its entirety.

While the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A method of characterizing a protein, the method comprising:
accessing simulated protein structure data with a computer system, wherein the simulated protein structure data indicate a structure of the protein;
using the computer system to:
a) select a plurality of residues in the protein and to quantify a plurality of dynamics metrics corresponding to each residue in the plurality of residues, wherein the dynamics metrics comprise at least a metric of epistasis,
wherein the metric of epistasis for a given residue in the plurality of residues is indicative of coupling additivity or non-additivity between the given residue and a pair of residues that does not comprise the given residue;
b) identify at least one residue of interest based on the plurality of dynamics metrics; and
c) characterize the protein and the at least one residue of interest based on the plurality of dynamics metrics.

2. The method of claim 1, wherein the plurality of dynamics metrics further comprises at least one of a dynamic flexibility index, a dynamic coupling index, or an asymmetric dynamic coupling index.

3. The method of claim 1, wherein quantifying the metric of epistasis for the given residue in the plurality of residues comprises:
perturbing each residue in the pair of residues individually and quantifying a response in the given residue;
perturbing each residue in the pair of residues simultaneously and quantifying the response in the given residue; and
calculating the metric of epistasis based on the responses to the individual and the simultaneous perturbations.

4. The method of claim 1, wherein the method further comprises:
accessing evolutionary information of the protein;
calculating an evolutionary probability score for each residue in the plurality of residues; and
identifying at least one candidate adaptive polymorphism in the plurality of residues based on the evolutionary probability scores.

5. The method of claim 4, wherein the method further comprises comparing the at least one residues of interest and the at least one candidate adaptive polymorphism to determine mechanistic or epistatic relationships between the at least one candidate adaptive polymorphism and the at least one residue of interest.

6. The method of claim 1, wherein the protein comprises a protein from a virus.

7. The method of claim 1, wherein the method further comprises:
repeating steps a) through c) on a protein variant; and
comparing the characterization of the protein and the at least one residue of the interest of the protein with the characterization of the protein variant and at least one residue of interest of the protein variant.

8. The method of claim 7, wherein the method further comprises predicting a functional behavior of the virus based on the plurality of dynamics metrics of each residue in the plurality of residues, and based on the at least one residue of interest.

9. A system for characterizing a protein, the system comprising:
a processor in communication with a memory, the memory having stored thereon a set of instructions which, when executed by the processor, cause the processor to:
a) access simulated protein structure data with a computer system, wherein the simulated protein structure data indicate a structure of the protein;
b) select a plurality of residues in the protein and to quantify a plurality of dynamics metrics corresponding to each residue in the plurality of residues, wherein the dynamics metrics comprise at least a metric of epistasis,
wherein the metric of epistasis for a given residue in the plurality of residues is indicative of coupling additivity or non-additivity between the given residue and a pair of residues that does not comprise the given residue;
c) identify at least one residue of interest based on the plurality of dynamics metrics; and
d) characterize the protein and the at least one residue of interest based on the plurality of dynamics metrics.

10. The system of claim 9, wherein the plurality of dynamics metrics further comprises at least one of a dynamic flexibility index, a dynamic coupling index, or an asymmetric dynamic coupling index.

11. The system of claim 9, wherein quantifying the metric of epistasis for the given residue in the plurality of residues comprises:
perturbing each residue in the pair of residues individually and quantifying a response in the given residue;
perturbing each residue in the pair of residues simultaneously and quantifying the response in the given residue; and
calculating the metric of epistasis based on the responses to the individual and the simultaneous perturbations.

12. The system of claim 9, wherein the system further comprises executing instructions to:
access evolutionary information of the protein;
calculate an evolutionary probability score for each residue in the plurality of residues; and
identify at least one candidate adaptive polymorphism in the plurality of residues based on the evolutionary probability scores.

13. The system of claim 12, wherein the system further comprises executing instructions to compare the at least one residue of interest and the at least one candidate adaptive polymorphism to determine mechanistic or epistatic relationships between the at least one candidate adaptive polymorphism and the at least one residue of interest.

14. The system of claim 9, wherein the protein comprises a protein from a virus.

15. The method of claim 14, wherein the system further comprises:
executing instructions to compare the characterization of the protein and the at least one residue of interest for the protein and the protein from the virus variant.

16. The system of claim 15, wherein the system further comprises executing instructions to predict a functional behavior of the virus based on the plurality of dynamics metrics of each residue in the plurality of residues, and based on the at least one residue of interest.

17. A non-transitory computer-readable medium having stored thereon instructions that, when executed by a processor, cause the processor to:
a) access simulated protein structure data with a computer system, wherein the simulated protein structure data indicate a structure of the protein;
b) select a plurality of residues in the protein and to quantify a plurality of dynamics metrics corresponding to each residue in the plurality of residues, wherein the dynamics metrics comprise at least a metric of epistasis,
wherein the metric of epistasis for a given residue in the plurality of residues is indicative of coupling additivity or non-additivity between the given residue and a pair of residues that does not comprise the given residue;
c) identify at least one residue of interest based on the plurality of dynamics metrics; and
d) characterize the protein and the at least one residue of interest based on the plurality of dynamics metrics.

18. The non-transitory computer-readable medium of claim 17, wherein the plurality of dynamics metrics further comprises at least one of a dynamic flexibility index, a dynamic coupling index, or an asymmetric dynamic coupling index.

19. The non-transitory computer-readable medium of claim 17, wherein quantifying the metric of epistasis for the given residue in the plurality of residues comprises:
perturbing each residue in the pair of residues individually and quantifying a response in the given residue;
perturbing each residue in the pair of residues simultaneously and quantifying the response in the given residue; and
calculating the metric of epistasis based on the responses to the individual and the simultaneous perturbations.

20. The non-transitory computer-readable medium of claim 17, wherein the non-transitory computer-readable medium further causes the processor to:
access evolutionary information of the protein;
calculate an evolutionary probability score for each residue in the plurality of residues; and
identify at least one candidate adaptive polymorphism in the plurality of residues based on the evolutionary probability scores.
